(19) United States
(12) Patent Application Publication
(10) Pub. No.: US 2023/0248658 A1
(43) Pub. Date: Aug. 10, 2023

(54) SUPRAMOLECULAR NANOSUBSTRATE-MEDIATED DELIVERY SYSTEM ENABLES CRISPR/CAS9 KNOCKIN OF HEMOGLOBIN BETA GENE-A POTENTIAL THERAPEUTIC SOLUTION FOR HEMOGLOBINOPATHIES

(71) Applicant: The Regents of the University of California, Oakland, CA (US)

(72) Inventors: Hsian-Rong Tseng, Oakland, CA (US); Peng Yang, Oakland, CA (US); Qian Ban, Oakland, CA (US)

(73) Assignee: The Regents of the University of California, Oakland, CA (US)

(21) Appl. No.: 17/776,523

(22) PCT Filed: Nov. 13, 2020
(86) PCT No.: PCT/US2020/060535

§ 371 Date: May 12, 2022

Related U.S. Application Data
(60) Provisional application No. 62/935,877, filed on Nov. 15, 2019.

(51) Int. Cl. A61K 9/51; C12N 9/22; C07K 14/805; C12N 15/90

(52) U.S. Cl. CPC A61K 9/5146; C07K 14/805; C12N 9/22; C12N 15/90; B82Y 5/00

(57) ABSTRACT

Compositions, systems and methods for delivering CRISPR/Cas9-based genome editing system and a donor protein to a cell.

### CROSS-REFERENCE OF RELATED APPLICATION

This application claims priority to U.S. Provisional Application No. 62/935,877 filed Nov. 15, 2019; the entire contents of all of which are hereby incorporated by reference.

### BACKGROUND

### 1. Technical Field

The field of the currently claimed embodiments of this invention relates to compositions, systems and methods for delivering CRISPR/Cas9-based genome editing system and a donor protein to a cell.

### 2. Discussion of Related Art

Hemoglobinopathies are a group of inherited genetic disorders caused by hemoglobin beta (HBB) gene mutations (1). Adult hemoglobin (HbA) consists of a tetramer of two α-globin and two β-globin subunits (α₂β₂). Several mutations in the HBB gene have been identified, which are associated with either structural changes or reduced production of β-globin protein, resulting in either altered hemoglobin production or function. These alterations are observed in the two most prevalent forms of β-hemoglobinopathies, i.e., sickle cell disease (SCD) and β-thalassemia (2). For example, SCD is caused by an A-to-T point mutation in the HBB gene, which leads to a substitution of a valine for a glutamic acid at position 6 in the β-globin chain. In addition, over 400 mutations in the HBB gene have been identified as associated with β-thalassemia. Traditionally, patients with severe hemoglobinopathy phenotypes require lifelong supportive care, which can include frequent red blood cell (RBC) transfusions (3). Allogeneic hematopoietic stem cell (HSC) transplantation (4), represents the only curative therapy for these patients. However, the lack of availability of well-matched donors to avoid transplant-associated co-morbidities, such as graft-versus-host disease, remains a critical challenge to achieving the best treatment outcomes. Gene therapies are emerging as promising alternative options for patients lacking an allogenic compatible HSC donor. Lentiviral vectors (LVs) carrying genetic materials encoding for either a corrected HBB gene or modifying regulators of hemoglobin production are now being applied clinically to enable effective autologous gene-modified HSC transplantation strategies (5). However, these LV-based gene addition approaches require correction of long-term repopulating HSCs and are extremely expensive to manufacture. Furthermore, LVs are limited by the size and type of cargo that they can deliver and may integrate semi-randomly into the genome, generating aberrant transcripts that may potentially trigger oncogenesis (5). Compared with LV-based gene transduction, genome-editing technologies offer a more practical approach, because they are designed to modify a single, safe genomic target (6).

The CRISPR/Cas9 genome editing system is evolving from an already powerful genetic editing research tool to a promising technology for treating genetic diseases (7). The CRISPR/Cas9 system is composed of two functional components, i.e., the Cas9 endonuclease and an engineered short, single-guide RNA (sgRNA), which form a ribonucleoprotein (RNP) complex, Cas9•sgRNA. Based on simple base-pairing, the Cas9•sgRNA complex recognizes and cuts at a target site pre-determined by the design of the sgRNA, introducing a precise double-stranded break (DSB) (7). Following the formation of a DSB, endogenous DNA repair can occur via either the i) non-homologous end joining (NHEJ) or ii) homology-directed repair (HDR) pathway, offering two classes of therapeutic genome editing approaches. In contrast to the NHEJ pathway, which serves as the foundation of CRISPR/Cas9-mediated gene knockdown and knockout, the HDR pathway enables CRISPR/Cas9-mediated gene correction and knockin, which can integrate a single-copy of a therapeutic gene at a pre-determined target site via a homologous donor DNA (dDNA) template, offering a more general therapeutic solution for a variety of genetic diseases (7). However, significant challenges remain for developing highly efficient methods for step-by-step delivery of CRISPR/Cas9 genome editing components into diseased cells, such as limited cargo size, different charge properties of CRISPR cargos, low delivery and genome editing efficiencies, and manufacturing challenges (8).

Physical methods, such as electroporation and microinjection, have been used successfully for delivering CRISPR/Cas9 reagents intracellularly via transient disruption of the lipid bilayer of cells (9). However, decreased cell viability and premature differentiation of the engineered stem cell product limit their clinical applications. Viral-based methods remain a popular choice for the delivery of gene-editing machinery, with adeno-associated virus (AAV) being the most promising viral vector (10). However, limitations in packaging capacity (<4.7 kb) (11), high cost, and safety concerns related to immunogenicity associated with AAV remain. Significant research has been devoted to developing less expensive and safer non-viral vectors (9, 12, 13) for delivering CRISPR/Cas9 reagents intracellularly, including lipids, polymers, and nanoparticles. The CRISPR/Cas9 system can be introduced in three forms: DNA (14), mRNA (15), and protein (16). Compared to deliveries of Cas9 DNA and Cas9 mRNA, direct delivery of Cas9•sgRNA has two major advantages: i) rapid genome editing, as it skips gene transcription and/or translation; and ii) transient genome editing with reduced off-target effects and toxicity. Due to the large size of the Cas9 protein (~160 kDa), there is a need for more effective delivery vectors (12). Lee et al. developed a delivery vehicle (CRISPR-Gold) composed of gold nanoparticles conjugated to DNA and complexed with cationic endosomal disruptive polymers to deliver CRISPR RNPs and donor template to correct the DNA mutation that causes DMD in mice with reduced off-target effects and reduced muscle fibrosis in mdx mice (17). Recently, Cheng et al. reported a strategy termed selective organ targeting (SORT) wherein multiple classes of lipid nanoparticles are systematically engineered to edit extrahepatic tissues exclusively via addition of a supplemental SORT molecule. Lung-, spleen-, and liver-targeted SORT lipid nanoparticles were designed to edit therapeutically relevant cell types selectively including epithelial cells, endothelial cells, B cells, T cells, and hepatocytes (18). However, most non-viral vector-based CRISPR/Cas9 genome editing systems have been developed for gene knockout (19), knockdown (20), or correction (21) to treat monogenic disorders. There has been relatively limited progress made in gene knockin (22), especially for achieving effective knockin of long-frame therapeutic genes in hematopoietic stem cells, which are notoriously difficult to transfect.

There thus remains a need for improved compositions, systems and methods for delivering and using CRISPR/Cas9.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a composition for delivering an endonuclease and a nucleic acid sequence encoding a donor protein to a cell, including: a first plurality of self-assembled supramolecular nanoparticles (SMNPs), each of the first plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, where the plurality of binding components and the plurality of cores self-assemble when brought into contact to form the first plurality of self-assembled supramolecular nanoparticles (SMNPs); a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the endonuclease; and a nucleotide sequence including a recognition sequence specific to the endonuclease; and a second plurality of self-assembled supramolecular nanoparticles (SMNPs), each of the second plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, where the plurality of binding components and the plurality of cores self-assemble when brought into contact to form the second plurality of self-assembled supramolecular nanoparticles (SMNPs); a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein. In such an embodiment, the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

An embodiment of the invention relates to a system for delivering an endonuclease and a nucleic acid sequence encoding a donor protein to a cell including: a first plurality of self-assembled supramolecular nanoparticles (SMNPs), each of the first plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, where the plurality of binding components and the plurality of cores self-assemble when brought into contact to form the first plurality of self-assembled supramolecular nanoparticles (SMNPs); a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the endonuclease; and a nucleotide sequence including a recognition sequence specific to the endonuclease; a second plurality of self-assembled supramolecular nanoparticles (SMNPs), each of the second plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, where the plurality of binding components and the plurality of cores self-assemble when brought into contact to form the second plurality of self-assembled supramolecular nanoparticles (SMNPs); a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein; and a device for capturing the cell, the device including: a substrate; and a plurality of nanowires at least one of attached to or integral with a surface of the substrate such that each nanowire of the plurality of nanowires has an unattached end. In such an embodiment, the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

An embodiment of the invention relates to a method for delivering an endonuclease and a nucleic acid sequence encoding a donor protein to a cell including: providing a first plurality of self-assembled supramolecular nanoparticles (SMNPs); providing a second plurality of self-assembled SMNPs; contacting the cell with at least one of the first plurality of self-assembled SMNPs, such that the at least one of the first plurality of self-assembled SMNPs is taken up by the cell; and contacting the cell with at least one of the second plurality of self-assembled SMNPs, such that the at least one of the second plurality of self-assembled SMNPs is taken up by the cell. In such an embodiment, each of the first plurality of self-assembled supramolecular nanoparticles SMNPs includes: a plurality of binding components, each having a plurality of binding regions; a plurality of cores that are suitable to at least provide some mechanical structure to the plurality of self-assembled SMNPs, the plurality of cores including at least one core binding element adapted to bind to the binding regions to form a first inclusion complex, where the plurality of binding components and the plurality of cores self-assemble when brought into contact to form the first plurality of self-assembled SMNPs; a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the endonuclease; and a nucleotide sequence including a recognition sequence specific to the endonuclease. In such an embodiment, each of the second plurality of self-assembled SMNPs includes: a plurality of binding components, each having a plurality of binding regions; a plurality of cores that are suitable to at least provide some mechanical structure to the plurality of self-assembled SMNPs, the plurality of cores including at least one core binding element adapted to bind to the binding regions to form a first inclusion complex, where the plurality of binding components and the plurality of cores self-assemble when brought into contact to form the second plurality of self-assembled SMNPs; a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein. In such an embodiment, the nucleic acid sequence encoding the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

### BRIEF DESCRIPTION OF THE DRAWINGS

Further objectives and advantages will become apparent from a consideration of the description, drawings, and examples.

FIGS. 1A-1C are illustrations showing a schematic for delivery of self-assembling supramolecular nanoparticles (SMNPs) to a cell, and illustrations showing the compositions of the SMNPs according to an embodiment of the invention.

FIGS. 2A-2D are illustrations and bar graphs showing the combined use of SMNPs and a nanowire system for delivery of SMNPs to captures cells according to an embodiment of the invention.

FIGS. 3A-3I are electron microscopy images of nanowires and nanoparticles according to embodiments of the invention.

FIGS. 4A-4D are illustrations, bar graphs and images showing optimization of a combined nanoparticle and nanowire approach to delivering CRISPR/Cas9 and a donor gene to a cell according to an embodiment of the invention.

FIGS. 5A-5F are images and schematics showing integration of a donor gene into a cell’s genomic DNA according to an embodiment of the invention.

FIGS. 6A-6H are images and schematics showing in vivo integration of a donor gene into a cell’s genomic DNA according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

An embodiment of the invention relates to a method employing the combined use of non-viral vectors with a homology directed repair (HDR) pathway to facilitate CRISPR/Cas9-mediated knockin of a full-length therapeutic gene or fragment thereof as a more effective and general non-viral therapeutic solution for many genetic diseases.

Some embodiments of the invention relate to compositions and methods for delivering a nucleic acid encoding an endonuclease and a nucleic acid sequence encoding a donor protein to a cell. In such embodiments, the composition includes a first plurality of self-assembled supramolecular nanoparticles (SMNPs) and a second plurality of self-assembled supramolecular nanoparticles (SMNPs). In such an embodiment, the nucleic acid encoding an endonuclease is encapsulated within each of the first plurality of self-assembled SMNPs, and the nucleic acid sequence encoding the donor protein is encapsulated within each of the second plurality of self-assembled SMNPs.

Some embodiments of the invention relate to compositions and methods for delivering an endonuclease and a donor protein to a cell. In such embodiments, the composition includes a first plurality of self-assembled SMNPs and a second plurality of self-assembled SMNPs. In such an embodiment, the endonuclease is encapsulated within each of the first plurality of self-assembled SMNPs, and the donor protein is encapsulated within each of the second plurality of self-assembled SMNPs.

As used throughout, the terms “donor protein” or “therapeutic protein” are used interchangeably and refer to a full length protein or fragment thereof serving as a functional replacement for a mutated or otherwise defective version of the protein or related gene endogenous to a cell or subject suffering from a genetic disorder associated with the mutated or defective protein or related gene. In some embodiments, the donor protein or fragment thereof is delivered into a cell or subject so that the donor protein or fragment thereof serves as a functional replacement for a mutated or otherwise defective version of the protein or related gene endogenous to the cell or subject.

In some embodiments, the donor protein or fragment thereof is delivered to a cell or subject in the form of a nucleic acid sequence encoding the donor protein or fragment thereof. In some embodiments, the nucleic acid sequence encoding the donor protein or fragment thereof is configured for insertion into the genomic DNA (gDNA) of a cell by homologous or non-homologous recombination. In some such embodiments, recombination of the nucleic acid sequence encoding the donor protein or fragment thereof into the gDNA of the host cell enables translation of donor protein or fragment thereof via the use of the cell’s machinery.

In some embodiments, the nucleic acid sequence encoding the donor protein or fragment thereof forms part of a circular, double-stranded DNA molecule (e.g. a plasmid) and is encapsulated in a supra-molecular nanoparticle configured for delivery of the nucleic acid sequence encoding the donor protein or fragment and circular, double-stranded DNA molecule into a cell. Methods of preparing a plasmid including a nucleic acid sequence encoding a donor protein or fragment thereof are known in the art.

In some embodiments, a plasmid including a nucleic acid sequence encoding a donor protein or fragment thereof is encapsulated in a self-assembled SMNPs configured for delivery of the plasmid into a cell. In some such embodiments, the nucleic acid sequence encoding the donor protein or fragment thereof is configured for insertion into the gDNA of a cell by homologous or non-homologous recombination. In some such embodiments, the nucleic acid sequence encoding the donor protein or fragment thereof is configured for insertion into the gDNA of a cell specifically by non-homologous recombination.

In some embodiments, a nucleic acid sequence encoding a donor protein or fragment thereof is configured for insertion into the gDNA of a cell by a CRISPR/Cas9-mediated system and by non-homologous recombination. In such an embodiment, the CRISPR/Cas9-mediated gene editing system is composed of two functional components, i.e., Cas9 endonuclease and an engineered short, single-guide RNA (sgRNA). Based on a base-pairing mechanism, Cas9·sgRNA complex recognizes and cuts a targeted site, precisely inducing a double-strand break (DSB). In some embodiments, endogenous DNA repair then occurs via a non-homologous end joining (NHEJ) pathway. The nucleic acid sequence encoding the donor protein or fragment thereof is then integrated into DSB.

In some embodiments, a nucleic acid sequence encoding the donor protein or fragment thereof is configured for insertion into the genomic DNA of a cell by a CRISPR/Cas9-mediated system and by a homology-independent targeted integration (HITI) strategy, which was previously developed and based on the NHEJ pathway to enable robust knockin of non-dividing cells in vivo. In such embodiments, the HITI strategy introduces two pre-determined CRISPR/Cas9 target sites into the nucleic acid sequence. After cutting the targeted sites present in both the gDNA and in the nucleic acid sequence, the resulting three DSB sites undergoes endogenous DNA repair via the NHEJ pathway to achieve integration of the nucleic acid sequence into the gDNA.

Some embodiments of the invention relate to a method of treating a genetic disorder. In such embodiments, a nucleic acid sequence encoding a donor protein or fragment thereof is configured for insertion into the gDNA of a cell from a subject suffering from the genetic condition by a CRISPR/Cas9-mediated system. In some such embodiments, a nucleic acid sequence encoding Cas9 and a separate sgRNA are encapsulated in a first population of self-assembled SMNPs, and the nucleic acid sequence encoding a donor protein or fragment thereof is encapsulated in a second population of self-assembled SMNPs. In some such embodiments, both populations of self-assembled SMNPs are configured for uptake by cells from the subject. In some such embodiments, the cell(s) from the subject is contacted with both populations of self-assembled SMNPs such that at least one self-assembled SMNP from each population of self-assembled SMNPs is taken up by the cell(s). Then, the nucleic acid sequence encoding Cas9 is released from its SMNP and Cas9 is encoded. The Cas9 forms a complex with the sgRNA, and cuts a target site in the gDNA of the cell. The nucleic acid sequence encoding the donor protein or fragment thereof is also released from its SMNP, and is then integrated into the cell’s gDNA at the target site via a NHEJ pathway. Once integrated, the donor protein or fragment thereof is then expressed and serves as a functional replacement for the mutated or otherwise defective version of the protein or related gene endogenous to the cell.

Some embodiments, relate to the method of treating a genetic disorder discussed above, where the genetic disorder is a hemoglobinopathy. Non-limiting examples of such genetic diseases include sickle cell disease, beta thalassemia, hemophilia, and acute lymphocytic leukemia.

Some aspects of the invention include supramolecular nanoparticles (SMNPs), having a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; and a plurality of terminating components, each having a single terminating binding element that binds to remaining binding regions of one of the plurality of binding components by forming a second inclusion complex. SMNPs are described in in U.S. Pat. No. 9845237 and U.S. Pat. Application No. 20160000918, each of which is herein incorporated in its entirety by reference. The plurality of binding components, plurality of cores, and the plurality of terminating components self-assemble when brought into contact to form the supramolecular magnetic nanoparticle (SMNP).

The plurality of binding components, plurality of cores, and the plurality of terminating components bind to each other by one or more intermolecular forces. Examples of intermolecular forces include hydrophobic interactions, biomolecular interactions, hydrogen bonding interactions, π-π interactions, electrostatic interactions, dipole-dipole interactions, or van der Waals forces. Examples of biomolecular interactions include DNA hybridization, a protein-small molecule interaction (e.g. protein-substrate interaction (e.g. a streptavidin-biotin interaction) or protein-inhibitor interaction), an antibody-antigen interaction or a protein-protein interaction. Examples of other interactions include inclusion complexes or inclusion compounds, e.g. adamantane-β-cyclodextrin complexes or diazobenzene-α-cyclodextrin complexes. Generally, the intermolecular forces binding the components of the SMNP structure are not covalent bonds.

Embodiments of the invention are related to compositions, systems, and/or methods for delivering CRISPR/Cas9-based genome editing system to a cell. In some embodiments, a self-assembled nano-particle is configured to encapsulate and deliver one or more functional Cas9 enzymes, Cpfl enzymes and one or more guide RNAs to a cell for editing of a genomic DNA sequence (including, but not limited to a gene, and intron, and/or and exon). In some embodiments, a self-assembled nano-particle is configured to encapsulate and deliver a nucleic acid sequence encoding for a Cas9 enzyme or a Cpfl enzyme. In some embodiments, a self-assembled nano-particle is configured to encapsulate and deliver a protein or peptide; non-limiting examples of such a protein or peptide include a recombinant protein or peptide, or a replacement protein or peptide. In some embodiments, a self-assembled nano-particle is configured to encapsulate and deliver a nucleotide sequence encoding a protein or a peptide.

Some embodiments of the invention are related to methods for genome editing in a cell. In some such embodiments, a target cell is contacted with a self-assembled nano-particle configured to encapsulate and deliver ne or more functional Cas9 enzymes, Cpfl enzymes and one or more guide RNAs to the cell for editing of a target genomic DNA sequence. In some embodiments, the self-assembled nano-particle is configured to encapsulate and deliver a nucleic acid sequence encoding for a Cas9 enzyme or a Cpfl enzyme. In some embodiments, the target cell is contacted with two different self-assembled nano-particles: a first self-assembled nano-particle configured to encapsulate and deliver to the cell a functional Cas9 enzyme and a guide RNA, or a nucleic acid sequence encoding for a Cas9 enzyme; and a second self-assembled nano-particle configured to encapsulate and deliver a protein or peptide or a nucleic acid sequence encoding a protein or peptide.

Some embodiments of the invention include a device for capturing a cell. Examples of such devices are described in U.S. Pat. No. 9140697, which is hereby incorporated by references in its entirety. A further non-limiting example of such a device is a Silicon Nanowire Substrates (SiNWS). In embodiments of the invention, the device includes a substrate; and a plurality of nanowires at least one of attached to or integral with a surface of the substrate such that each nanowire of the plurality of nanowires has an unattached end. In some embodiments, the plurality of nanowires are configured to reversibly attach to self-assembled supramolecular nanoparticles (SMNPs).

In some embodiment, the device for capturing a cell includes a substrate having a nanostructured surface region. Also, in some embodiments, a plurality of binding agents are attached to the nanostructured surface region of the substrate. However, binding agents are not required for the device to bind to target cells. The nanostructured surface region includes a plurality of nanostructures, each having a longitudinal dimension and a lateral dimension. As a sample is placed on the device, biological cells are selectively captured by the binding agents and the plurality of nanostructures acting in cooperation (in embodiments having binding agents). When present, the binding agent or agents employed will depend on the type of biological cell(s) being targeted. Conventional binding agents are suitable for use in some of the embodiments of the present invention. Non-limiting examples of binding agents include antibodies, nucleic acids, oligo- or polypeptides, cellular receptors, ligands, aptamers, biotin, avidin. Coordination complexes, synthetic polymers, and carbohydrates. In some embodiments of the present invention, binding agents are attached to the nanostructured surface region using conventional methods. The method employed will depend on the binding agents and the material used to construct the device. Non-limiting examples of attachment methods include non-specific adsorption to the surface, either of the binding agents or a compound to which the agent is attached or chemical binding, e.g., through self-assembled monolayers or silane chemistry. In some embodiments, the nanostructured surface region is coated with streptavidin and the binding agents are biotinylated, which facilitates attachment to the nanostructured surface region via interactions with the streptavidin molecules.

In some embodiments of the present invention, the nanostructures increase the surface area of the substrate and increase the probability that a given cell will come into contact. In these embodiments, the nanostructures can enhance binding of the target cells by interacting with cellular surface components such as microvilli, lamellipodia, filopodia, and lipid-raft molecular groups. In some embodiments, the nanostructures have a longitudinal dimension that is equal to its lateral dimension, where both the lateral dimension and the longitudinal dimension is less than 1 mm, i.e., nanoscale in size. In other embodiments, the nanostructures have a longitudinal dimension that is at least ten times greater than its lateral dimension. In further embodiments, the nanostructures have a longitudinal dimension that is at least twenty times greater, fifty times greater, or 100 times greater than its lateral dimension. In some embodiments, the lateral dimension is less than 1 mm. In other embodiments, the lateral dimension is between 1-500 nm. In further embodiments, the lateral dimension is between 30-400 nm. In still further embodiments, the lateral dimension is between 50-250 nm. In some embodiments, the longitudinal dimension is at least 1 mm long. In other embodiments, the longitudinal dimension is between 1-50 mm long. In other embodiments, the longitudinal dimension is 1-25 mm long. In further embodiments, the longitudinal dimension is 5-10 mm long. In still further embodiments, the longitudinal dimension is at least 6 mm long. The shape of the nanostructure is not critical. In some embodiments of the present invention, the nanostructure is a sphere or a bead. In other embodiments, the nanostructure is a strand, a wire, or a tube. In further embodiments, a plurality of nanostructure contains one or more of nanowires, nanofibers, nanotubes, nano-pillars, nanospheres, or nanoparticles.

Some embodiments of the invention include a composition having a plurality of self-assembled SMNPs, where the self-assembled SMNPs include a membrane penetration ligand. IN such embodiments, the membrane penetration ligand (e.g. TAT) is attached to the outer surface of the self-assembled SMNPs via in situ ligand dynamic exchange with adamantane-grafted polyethylene glycol (Ad-PEG) based on multivalent molecular recognition between b-cyclodextrin (CD) and adamantane (Ad) motifs. Non-limiting examples of concentrations or ratios of the Ad-PEG-TAT to Ad-PEG are from 1:100 to 50:100. Non-limiting examples of membrane penetrating ligands include TAT (GRKKRRQRRRPQ) (SEQ ID NO: 1), RGD (CRGDKGPDC) (SEQ ID NO:2), MPG (GLAFLGFLGAAGSTMGAWSQPKKKRKV) (SEQ ID NO:3), Pep-1 (KETW-WETWWTEWSQPKKRKV) (SEQ ID NO:4), GALA (WEAALAEALAEALAEHLAEALAEALEALAA) (SEQ ID NO: 5), MAP17 (QLALQLALQALQAALQLA) (SEQ ID NO:6), and MAP12(LKTLTETLKELTKTLTEL) (SEQ ID NO:7). Additional example membrane penetrating ligands would be apparent to one of ordinary skill in the art.

An embodiment of the invention relates to a composition for delivering an endonuclease and a nucleic acid sequence encoding a donor protein to a cell, including: a first plurality of self-assembled supramolecular nanoparticles (SMNPs), each of the first plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, where the plurality of binding components and the plurality of cores self-assemble when brought into contact to form the first plurality of self-assembled supramolecular nanoparticles (SMNPs); a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the endonuclease; and a nucleotide sequence including a recognition sequence specific to the endonuclease; and a second plurality of self-assembled supramolecular nanoparticles (SMNPs), each of the second plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, where the plurality of binding components and the plurality of cores self-assemble when brought into contact to form the second plurality of self-assembled supramolecular nanoparticles (SMNPs); a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein. In such an embodiment, the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

An embodiment of the invention relates to the composition above, where the endonuclease is a CRISPR associated protein 9 (Cas9), and where the nucleotide sequence is a single guide RNA (sgRNA).

An embodiment of the invention relates to the composition above, where the first plurality of self-assembled SMNPs and the endonuclease are present in a percent mass (w/w) ratio of between 100:1 to 100:50.

An embodiment of the invention relates to the composition above, where the second plurality of self-assembled SMNPs and the nucleic acid sequence encoding the donor protein are present in a percent mass (w/w) ratio of between 100:1 and 100:50.

An embodiment of the invention relates to the composition above, where each of the first plurality of self-assembled SMNPs and each of the second plurality of self-assembled SMNPs have a diameter of between 100 nanometers and 200 nanometers.

An embodiment of the invention relates to the composition above, where the first plurality of self-assembled SMNPs and the second plurality of SMNPs further include a membrane penetration ligand coupled to an outer surface.

An embodiment of the invention relates to the composition above, where the plurality of binding components of the first and second plurality of self-assembled SMNPs includes polythylenimine, poly(L-lysine), or poly(β-amino ester).

An embodiment of the invention relates to the composition above, where the plurality of binding regions of the first and second plurality of self-assembled SMNPs includes beta-cyclodextrin, alpha-cyclodextrin, gamma-cyclodextrin, cucurbituril or calixarene.

An embodiment of the invention relates to the composition above, where the plurality of cores of the first and second plurality of self-assembled SMNPs includes polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the composition above, where the at least one core binding element of the first and second plurality of self-assembled SMNPs includes adamantane, azobenzene, ferrocene or anthracene.

An embodiment of the invention relates to the composition above, where the plurality of terminating components of the first and second plurality of self-assembled SMNPs includes polyethylene glycol (PEG) or polypropylene glycol) (PGG).

An embodiment of the invention relates to the composition above, where the single terminating binding element of the first and second plurality of self-assembled SMNPs includes adamantane, azobenzene, ferrocene or anthracene.

An embodiment of the invention relates to a system for delivering an endonuclease and a nucleic acid sequence encoding a donor protein to a cell including: a first plurality of self-assembled supramolecular nanoparticles (SMNPs), each of the first plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, where the plurality of binding components and the plurality of cores self-assemble when brought into contact to form the first plurality of self-assembled supramolecular nanoparticles (SMNPs); a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the endonuclease; and a nucleotide sequence including a recognition sequence specific to the endonuclease; a second plurality of self-assembled supramolecular nanoparticles (SMNPs), each of the second plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, where the plurality of binding components and the plurality of cores self-assemble when brought into contact to form the second plurality of self-assembled supramolecular nanoparticles (SMNPs); a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein; and a device for capturing the cell, the device including: a substrate; and a plurality of nanowires at least one of attached to or integral with a surface of the substrate such that each nanowire of the plurality of nanowires has an unattached end. In such an embodiment, the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

An embodiment of the invention relates to the system above, where at least one of the first plurality of self-assembled SMNPs and the second plurality of self-assembled SMNPs are configures to reversibly attach to the plurality of nanowires.

An embodiment of the invention relates to the system above, where the endonuclease is a CRISPR associated protein 9 (Cas9), and where the nucleotide sequence is a single guide RNA (sgRNA).

An embodiment of the invention relates to the system above, where the first plurality of self-assembled SMNPs and the endonuclease are present in a percent mass (w/w) ratio of between 100:1 to 100:50.

An embodiment of the invention relates to the system above, where the second plurality of self-assembled SMNPs and the nucleic acid sequence encoding the donor protein are present in a percent mass (w/w) ratio of between 100:1 and 100:50.

An embodiment of the invention relates to the system above, where each of the first plurality of self-assembled SMNPs and each of the second plurality of self-assembled SMNPs have a diameter of between 100 nanometers and 200 nanometers.

An embodiment of the invention relates to the system above, where the first plurality of self-assembled SMNPs and the second plurality of SMNPs further include a membrane penetration ligand coupled to an outer surface.

An embodiment of the invention relates to the system above, where the plurality of binding components of the first and second plurality of self-assembled SMNPs includes polythylenimine, po]y(L-lysine), or poly(β-amino ester).

An embodiment of the invention relates to the system above, where the plurality of binding regions of the first and second plurality of self-assembled SMNPs includes beta-cyclodextrin, alpha-cyclodextrin, gamma-cyclodextrin, cucurbituril or calixarene.

An embodiment of the invention relates to the system above, where the plurality of cores of the first and second plurality of self-assembled SMNPs includes polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the system above, where the at least one core binding element of the first and second plurality of self-assembled SMNPs includes adamantane, azobenzene, ferrocene or anthracene.

An embodiment of the invention relates to the system above, where the plurality of terminating components of the first and second plurality of self-assembled SMNPs includes polyethylene glycol (PEG) or polypropylene glycol) (PGG).

An embodiment of the invention relates to the system above, where the single terminating binding element of the first and second plurality of self-assembled SMNPs includes adamantane, azobenzene, ferrocene or anthracene.

An embodiment of the invention relates to the system above, where the plurality of nanowires has a diameter of between 50 nanometers and 100 nanometers.

An embodiment of the invention relates to the system above, where the plurality of nanowires has a length of between 5 micrometers and 10 micrometers.

An embodiment of the invention relates to the system above, where the plurality of nanowires includes silicon, gold, silver, SiO2, or TiO2.

An embodiment of the invention relates to a method for delivering an endonuclease and a nucleic acid sequence encoding a donor protein to a cell including: providing a first plurality of self-assembled supramolecular nanoparticles (SMNPs); providing a second plurality of self-assembled SMNPs; contacting the cell with at least one of the first plurality of self-assembled SMNPs, such that the at least one of the first plurality of self-assembled SMNPs is taken up by the cell; and contacting the cell with at least one of the second plurality of self-assembled SMNPs, such that the at least one of the second plurality of self-assembled SMNPs is taken up by the cell. In such an embodiment, each of the first plurality of self-assembled supramolecular nanoparticles SMNPs includes: a plurality of binding components, each having a plurality of binding regions; a plurality of cores that are suitable to at least provide some mechanical structure to the plurality of self-assembled SMNPs, the plurality of cores including at least one core binding element adapted to bind to the binding regions to form a first inclusion complex, where the plurality of binding components and the plurality of cores self-assemble when brought into contact to form the first plurality of self-assembled SMNPs; a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the endonuclease; and a nucleotide sequence including a recognition sequence specific to the endonuclease. In such an embodiment, each of the second plurality of self-assembled SMNPs includes: a plurality of binding components, each having a plurality of binding regions; a plurality of cores that are suitable to at least provide some mechanical structure to the plurality of self-assembled SMNPs, the plurality of cores including at least one core binding element adapted to bind to the binding regions to form a first inclusion complex, where the plurality of binding components and the plurality of cores self-assemble when brought into contact to form the second plurality of self-assembled SMNPs; a plurality of terminating components, each having a single terminating binding element that binds to remaining binding regions of one of the plurality of binding components by forming a second inclusion complex, where the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein. In such an embodiment, the nucleic acid sequence encoding the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

An embodiment of the invention relates to the method above, where the endonuclease is a CRISPR associated protein 9 (Cas9), and the nucleotide sequence is a single guide RNA (sgRNA).

An embodiment of the invention relates to the method above, where contacting the cell with at least one of the second plurality of self-assembled SMNPs is carried out between 1 to 12 hours after the contacting the cell with at least one of the first plurality of self-assembled SMNPs.

An embodiment of the invention relates to the method above, where contacting the cell with at least one of the second plurality of self-assembled SMNPs is carried out between 1 to 12 hours after the contacting the cell with at least one of the first plurality of self-assembled SMNPs is repeated.

An embodiment of the invention relates to the method above, further including contacting the cell with a device for capturing the cell prior to the contacting the cell with at least one of the first plurality of self-assembled SMNPs, where the device for capturing the cell, the device includes: a substrate; and a plurality of nanowires at least one of attached to or integral with a surface of the substrate such that each nanowire of the plurality of nanowires has an unattached end.

An embodiment of the invention relates to the method above, where the first plurality of self-assembled SMNPs and the endonuclease are present in a percent mass (w/w) ratio of between 100:1 to 100:50.

An embodiment of the invention relates to the method above, where the second plurality of self-assembled SMNPs and the nucleic acid sequence encoding the donor protein are present in a percent mass (w/w) ratio of between 100:1 and 100:50.

An embodiment of the invention relates to the method above, where each of the first plurality of self-assembled SMNPs and each of the second plurality of self-assembled SMNPs have a diameter of between 100 nanometers and 200 nanometers.

An embodiment of the invention relates to the method above, where the first plurality of self-assembled SMNPs and the second plurality of SMNPs further include a membrane penetration ligand coupled to an outer surface.

An embodiment of the invention relates to the method above, where the plurality of binding components of the first and second plurality of self-assembled SMNPs includes polythylenimine, poly(L-lysine), or poly(β-amino ester).

An embodiment of the invention relates to the method above, where the plurality of binding regions of the first and second plurality of self-assembled SMNPs includes beta-cyclodextrin, alpha-cyclodextrin, gamma-cyclodextrin, cucurbituril or calixarene.

An embodiment of the invention relates to the method above, where the plurality of cores of the first and second plurality of self-assembled SMNPs includes polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the method above, where the at least one core binding element of the first and second plurality of self-assembled SMNPs includes adamantane, azobenzene, ferrocene or anthracene.

An embodiment of the invention relates to the method above, where the plurality of terminating components of the first and second plurality of self-assembled SMNPs includes polyethylene glycol (PEG) or polypropylene glycol) (PGG).

An embodiment of the invention relates to the method above, where the single terminating binding element of the first and second plurality of self-assembled SMNPs includes adamantane, azobenzene, ferrocene or anthracene.

### Examples

### Example 1

Polyamidoamine (PAMAM) dendrimer is a kind of hyperbranched polymer with high molecular uniformity, narrow molecular weight distribution, defined size, and an amine terminal surface. These properties enable it to be modified with different numbers of adamantane (Ad) motifs to prepare adamantane-grafted polyamidoamine (Ad-PAMAM) dendrimers. Previously, we demonstrated a convenient and flexible self-assembled synthetic approach for producing supramolecular nanoparticle (SMNP) vectors (23) by mixing three molecular building blocks, i.e., Ad-PAMAM dendrimer, beta-cyclodextrin (CD)-grafted branched polyethyleneimine (CD-PEI), and Ad-grafted poly(ethylene glycol) (Ad-PEG). Multivalent molecular recognition between Ad and CD enables modular control over the sizes, surface chemistry, and payloads of SMNP vectors, with a diversity of imaging (23) and therapeutic applications (24). To improve the delivery efficiency of SMNP vectors, we developed a substrate-mediated delivery strategy (25), a.k.a., supramolecular nanosubstrate-mediated delivery (SNSMD), by which Ad-grafted silicon nanowire substrates (Ad-SiNWS) were employed to facilitate the uptake of SMNP vectors into cells. The Ad/CD recognition system drives dynamic assembly and local enrichment of SMNPs onto Ad-SiNWS. Once the cells settle onto the Ad-SiNWS, intimate contact between the cell membrane and the nanowires leads to efficient delivery of SMNP vectors. We envision that the combined utility of SMNP vectors and SNSMD (i.e., a combined SMNP/SNSMD strategy) offers a powerful solution for step-by-step delivery of Cas9·sgRNA and dDNA for highly efficient genome editing in cells, especially for hematologic cells.

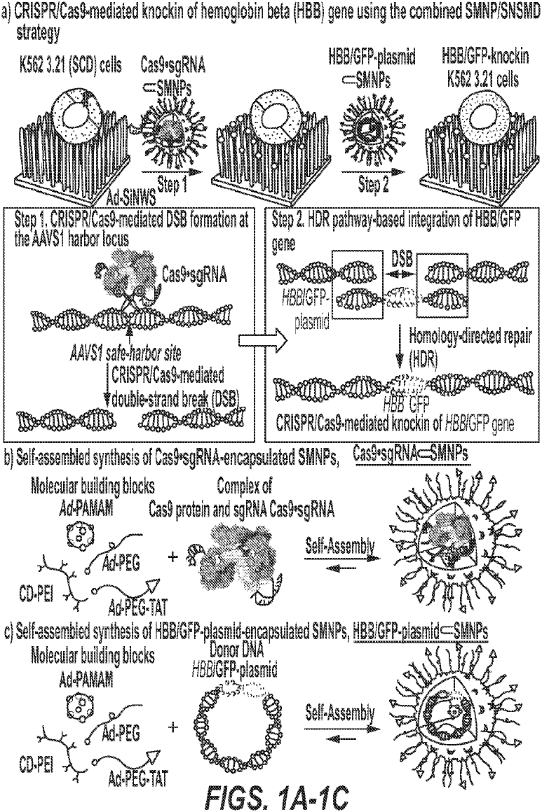

Here, a combined SMNP/SNSMD strategy is demonstrated (FIGS. 1A-1C) that enables the CRISPR/Cas9-mediated knockin of a single-copy HBB gene to the human adeno-associated virus integration site 1 (AAVS1) safe-harbor locus, providing a safe, effective, cheap, and general curative therapeutic solution for hemoglobinopathies. The K562 3.21 cell line has a known HBB mutation (associated with SCD) (26) and was employed as a model system for optimization and feasibility studies. Two different SMNP vectors, i.e., Cas9•sgRNA⊂SMNPs and HBB/GFP-plasmid⊂SMNPs, were prepared (FIGS. 1B and C) by encapsulating Cas9·sgRNA (targeting the AAVS1 site) and dDNA into SMNP vectors via a self-assembled synthetic approach, respectively. Here, CRISPR/Cas9 knockin of the HBB/GFP gene is carried out via two consecutive steps. In Step 1, the combined SMNP/SNSMD strategy facilitates cell uptake of Cas9•sgRNA⊂SMNPs, and the internalized and released Cas9·sgRNA specifically recognizes and induces DSB at the AAVS1 site. In Step 2, HBB/GFP-plasmid⊂SMNPs were added to deliver the HBB/GFP-plasmid, and the HDR pathway led to integration of the HBB/GFP gene into the DSB. We examined how the delivery time interval and multiple treatments of these two SMNP vectors affected HBB/GFP knockin efficiency. Under optimized step-by-step delivery conditions, HBB/GFP-knockin K562 3.21 cells were produced, sorted, and expanded. Fluorescence microscopy, polymerase chain reaction (PCR), and Sanger sequencing were employed to confirm the successful integration of the 3.7-kb HBB/GFP gene into the AAVS1 site in the cells. Furthermore, the HBB/GFP-knockin K562 3.21 cells were tested with immunofluorescence (IF) analysis and quantitative PCR assays, indicating that the integrated HBB gene was functionally expressed at both protein and mRNA levels, respectively. Finally, the HBB/GFP-knockin K562 3.21 cells were introduced into athymic nude mice via intraperitoneal injection to test their ability to proliferate and yield consistent HBB/GFP gene expression in vivo.

### Results

### Synthesis of EGFP-Cas9•sgRNA⊂SMNPs and Analysis of Cellular Uptake

Previous studies have used SMNP vectors for co-encapsulating a transcription factor (TF) protein and a DNA plasmid (27) to prepare Cas9•sgRNA⊂SMNPs (FIG. 1B).

FIGS. 1A-1C show results of CRISPR/Cas9-mediated knockin of hemoglobin beta/green fluorescent protein (HBB/GFP) gene. FIG. 1A is a schematic of the mechanism governing the combined supramolecular nanoparticle (SMNP)/supramolecular nanosubstrate-mediated delivery (SNSMD) strategy for CRISPR/Cas9-mediated knockin of HBB/GFP gene into K562 3.21 (SCD) cells via two consecutive steps. FIG. 1B shows a self-assembled synthetic approach for the preparation of Cas9•sgRNA⊂SMNPs through stoichiometric mixing of Cas9·sgRNA and the four molecular building blocks. FIG. 1C shows a self-assembled synthetic approach for the preparation of HBB/GFP-plasmid⊂SMNPs.

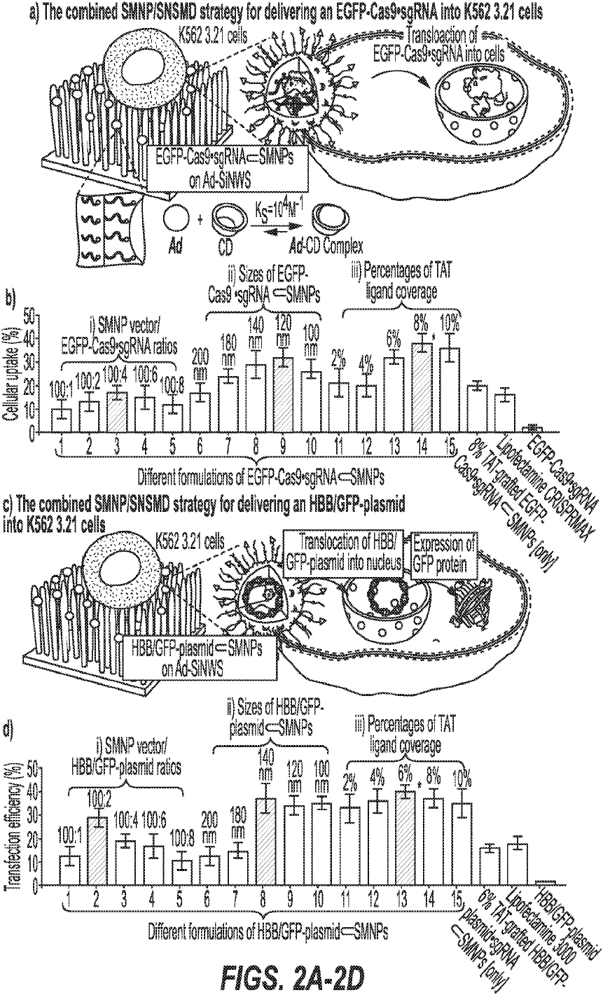

First, an sgRNA targeting the AAVS1 locus (28) was purchased (Integrated DNA Technologies, Iowa) for preparing Cas9•sgRNA. Cas9•sgRNA⊂SMNPs were prepared through stoichiometric mixing of Cas9·sgRNA and four SMNP molecular building blocks. In search of an optimal formulation of Cas9•sgRNA⊂SMNPs, EGFP-labeled Cas9 protein (EGFP-Cas9, GenCrispr, New Jersey) was used to study uptake into K562 3.21 cells (FIG. 2A). Three batches (15 formulations) of EGFP-Cas9•sgRN⊂SMNPs (FIG. 2B) were formulated by stepwise modulation of: i) the SMNP/EGFP-Cas9•sgRNA weight ratio (100:1, 100:2, 100:4, 100:6, and 100:8); ii) SMNP size (100-200 nm); and iii) the coverage of a membrane penetration ligand, TAT (2-10%). Prior to the cell uptake study, K562 3.21 cells were starved in serum-free RPMI medium for 10 h to synchronize their cell cycles to G0/G1 phases (29). Approximately 1×10⁵ K562 3.21 cells were introduced into each well of 8-well culture plates (10.5 cm²/well), in which an Ad-SiNWS (2.5×3.0 cm²) was immersed in 1.0 mL RPMI medium. Each formulation of EGFP-Cas9•sgRNA⊂SMNPs (containing 3.0 µg of EGFP-Cas9) was added to one well. After DAPI nuclear staining, the K562 cells on Ad-SiNWS were subjected to high-resolution microscopy imaging. First was examined how the weight ratios (wt%) between SMNP vectors and EGFP-Cas9•sgRNA affect cell uptake. The results (FIG. 2B) indicate that a higher percentage of EGFP-positive cells (17%) was achieved at the ratio of 100:4. Using this formulation ratio, next was studied how the sizes of Cas9•sgRNA⊂SMNPs affect cellular uptake. By altering the amount of Ad-PAMAM and CD-PEI in each formulation, the second batch of EGFP-Cas9•sgRNA⊂SMNPs was prepared with five different sizes, ranging from 110 to 200 nm. It was determined that the 120 nm EGFP-Cas9•sgRNA⊂SMNPs led to better delivery performance with 28% EGFP-positive cells. Based on this SMNP configuration, TAT-grafted EGFP-Cas9•sgRNA⊂SMNPs were prepared with TAT coverage ranging between 2% to 10%. These studies revealed that EGFP-Cas9•sgRNA⊂SMNPs with 8% TAT coverage exhibited an optimal delivery performance of 35%. The optimal synthetic formulation that gave 120 nm 8%-TAT-grafted EGFP-Cas9•sgRNA⊂SMNPs was determined, and this formulation was subjected to gene-editing studies. Three control studies, i.e., K562 3.21 cells treated with EGFP-Cas9•sgRNA⊂SMNPs (no Ad-SiNWS), Lipofectamine CRISPRMAX agent (encapsulated EGFP-Cas9•sgRNA) and EGFP-Cas9•sgRNA (without SMNP vectors) were conducted in parallel. The 8% TAT-grafted Cas9•sgRNA⊂SMNPs showed about half the uptake efficiency without nanowires. The EGFP-Cas9-sgRNA delivered with lipofectamine had a similar efficiency to the SMNPs without nanowires. EGFP-Cas9-sgRNA alone resulted in low efficiency. The dramatically compromised EGFP-Cas9 uptake performance highlights the critical roles of the two functional components, i.e., Ad-SiNWS and SMNP vectors, of the combined SMNP/SNSMD strategy. To visualize intracellular distribution and clearance of the SMNPs, CD-PEI-Cy5 instead of CD-PEI were used, to assemble the Cas9•sgRNA⊂Cy5-SMNPs in the uptake experiment. According to the fluorescent images, it appears that the Cas9•sgRNA⊂Cy5-SMNPs (red) were taken up by the K562 3.21 cells after incubation for 1 day. Thereafter, slow decay of red fluorescence intensity was observed from day 2 to day 7, suggesting the CD-PEI-Cy5 was cleared by the cells. To determine potential off-target effects, PCR and Sanger sequence were performed at five of the top-ranking predicted off-target sites of the sgRNA-AAVS1 via the Cas-OFFinder design tool. The sequencing results showed no detectable off-target events in the top 5 predicted off-target loci.

### Synthesis of HBB/GFP-Plasmid⊂SMNPs and Transfection Studies

Using a similar screening approach, the optimal formulation for preparing HBB/GFP-plasmid⊂SMNPs was identified (FIG. 2C). Three batches (15 formulations) of SMNPs were prepared by stepwise modulation of: i) the SMNP/plasmid weight ratios (100:1, 100:2, 100:4, 100:6, and 100:8); ii) SMNP size (100-200 nm); and iii) the coverage of TAT (2-10%). After settling growth-synchronized K562 3.21 cells on Ad-SiNWS in culture plates, individual formulations of the SMNPs (containing 2.0 µg of HBB/GFP-plasmid) were added to evaluate transfection of the GFP-encoding transgene. After treatment with SMNPs for 24 h, fluorescence microscopy was used to quantify the GFP expression in individual cells. The results revealed (FIG. 2D) that 140-nm HBB/GFP-plasmid⊂SMNPs with 6% TAT coverage exhibited an optimal GFP transfection performance of 38%. Three control studies, i.e., K562 3.21 cells treated with HBB/GFP-plasmid⊂SMNPs (no Ad-SiNWS), Lipofectamine 3000 agent (encapsulated HBB/GFP-plasmid) and HBB/GFP-plasmid (without SMNP vectors) were conducted in parallel. This HBB/GFP-plasmid⊂SMNP formulation was consequently subjected to time-dependent quantitative imaging at 12, 24, 48, 72, 96, and 120 h post treatment, showing that the highest transfection performance (45%) was observed 48 h post treatment, and the GFP signals gradually decayed and diminished completely by 120 h.

FIGS. 2A-2D show the combined SMNP/SNSMD strategy for delivering EGFP-Cas9•sgRNA or HBB/GFP-plasmid into K562 3.21 cells. FIG. 2A shows the combined SMNP/SNSMD strategy for delivering EGFP-Cas9•sgRNA into K562 3.21 cells. In FIG. 2B, three batches (15 formulations) of EGFP-Cas9•sgRNA⊂SMNPs were prepared for delivering EGFP-Cas9•sgRNA into K562 3.21 cells to identify an optimal formulation (marked with an asterisk). FIG. 2C shows the combined SMNP/SNSMD strategy for delivering HBB/GFP-plasmid into K562 3.21 cells. In FIG. 2D, three batches (15 formulations) of HBB/GFP-plasmid⊂SMNPs were prepared for GFP transfection studies to identify an optimal formulation (marked with an asterisk).

### Characterization of SMNPs and Ad-SiNWS

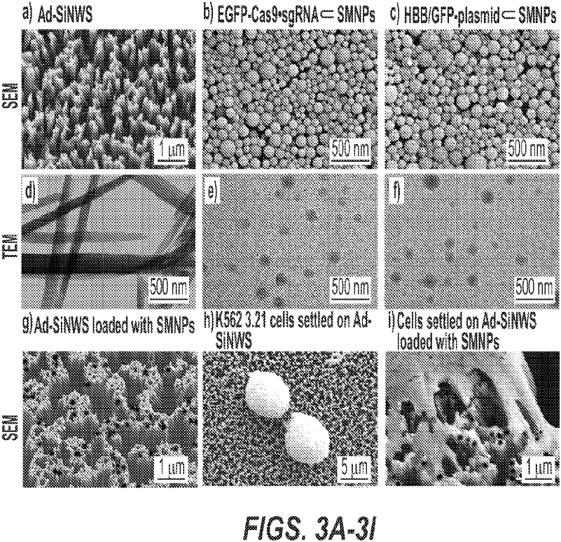

Scanning electron microscopy (SEM) and transmission electron microscopy (TEM) were used to characterize Ad-SiNWS, EGFP-Cas9•sgRNA⊂SMNPs, and HBB/GFP-plasmid⊂SMNPs, identified above. The SEM and TEM images showed that the diameters and lengths of Ad-SiNWS are ca. 50-100 nm and 5-10 µm, respectively (FIGS. 3A, D and S6A). EGFP-Cas9•sgRNA⊂SMNPs (FIGS. 3B and E) and HBB/GFP-plasmid⊂SMNPs (FIGS. 3C and F) showed homogeneous spherical morphologies with sizes of 108 ± 37 nm and 125 ± 43 nm, respectively (FIG. S6B). The surface-charge densities of SMNPs were determined by zeta potential measurements in phosphate buffered saline (PBS) buffer solution, which showed that the zeta potentials of 120 nm 8%-TAT-grafted Cas9•sgRNA⊂SMNPs and 140 nm 6% TAT-grafted HBB/GFP-plasmid⊂SMNPs were +26 ± 4 mV and +23 ± 5 mV, respectively. The assembly of SMNPs onto Ad-SiNWS (FIG. 3G) and the interactions between cells and Ad-SiNWS (FIGS. 3H and I) were also visualized by SEM, supporting the working mechanism of the SMNP/SNSMD strategy. According to stoichiometric calculations, it was estimated that ca. 130 Cas9•sgRNA complexes and 2-3 HBB/GFP-plasmids were encapsulated into each Cas9•sgRNA⊂SMNP and HBB/GFP-plasmid⊂SMNP under the optimal formulation conditions, respectively.

FIGS. 3A-3I are scanning electron microscopy (SEM) and transmission electron microscopy (TEM) images of SMNPs and of Ad-SiNWS. FIG. 3A shows SEM images of adamantane-grafted silicon nanowire substrates (Ad-SiNWS), FIG. 3B shows 8%-TAT-grafted EGFP-Cas9•sgRNA⊂SMNPs, and FIG. 3C shows 6%-TAT-grafted HBB/GFP-plasmid⊂SMNPs. FIG. 3D shows TEM images of free nanowires released from Ad-SiNWS, FIG. 3E shows EGFP-Cas9•sgRNA⊂SMNPs, and FIG. 3F shows HBB/GFP-plasmid⊂SMNPs. FIG. 3G shows SEM images of EGFP-Cas9•sgRNA⊂SMNPs Ad-SiNWS, FIG. 3H shows K562 3.21 cells settled onto Ad-SiNWS, and FIG. 3I shows a K562 3.21 cell settled on EGFP-Cas9•sgRNA⊂SMNPs grafted Ad-SiNWS.

### CRISPR/Cas9 Knockin of the HBB/GFP Gene in K562 3.21 Cells

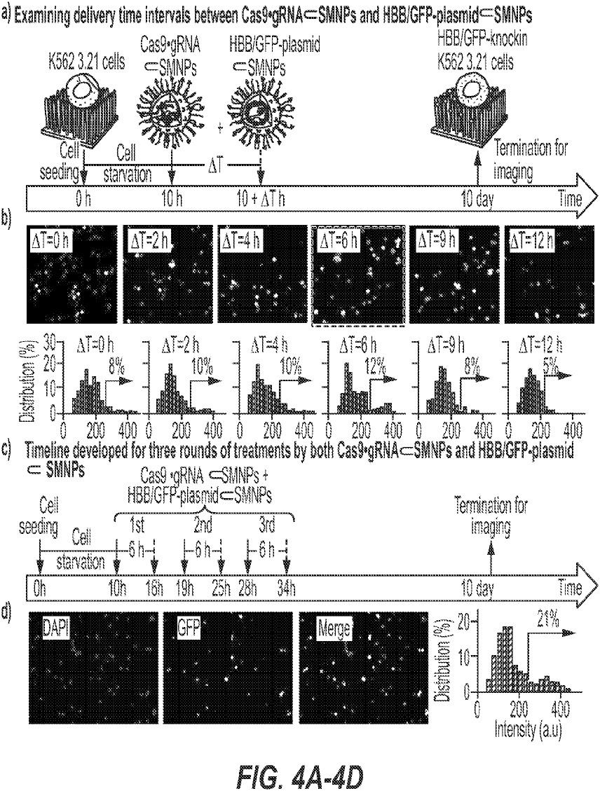

Next was studied the combined SMNP/SNSMD strategy for CRISPR/Cas9 knockin of the HBB/GFP gene in K562 3.21 cells in conjunction with the use of both 120 nm 8%-TAT-grafted Cas9•sgRNA⊂SMNPs and 140 nm 6% TAT-grafted HBB/GFP-plasmid⊂SMNPs obtained above. Considering the two consecutive steps associated with the CRISPR/Cas9 knockin process (FIG. 1A), it was hypothesized that the Cas9·sgRNA and donor HBB/GFP-plasmid should arrive in sequence to optimize knockin efficiency. To test this hypothesis, how the delivery time interval (ΔT = 0, 2, 4, 6, 9, or 12 h) between Cas9•sgRNA⊂SMNPs (containing 3.0 µg Cas9 protein) and HBB/GFP-plasmid⊂SMNPs (containing 2.0 µg HBB/GFP-plasmid) affected the knockin efficiency in growth-synchronized K562 3.21 cells was examined (FIG. 4A). The treated cells were maintained until termination at day 10, followed by fluorescence microscopy imaging to quantify the GFP signals. FIG. 4B compiles serial fluorescent micrographs of the resulting K562 3.21 cells, revealing that the highest HBB/GFP knockin efficiency (12%) was observed at ΔT = 6 h. Further, to take advantage of the combined SMNP/SNSMD strategy for serial delivery, three rounds of the SMNP treatments that ensure a steady supply of both Cas9·sgRNA and HBB/GFP-plasmid over a period of 24 h were conducted according to the timeline shown in FIG. 4C. Fluorescence microscopy imaging (FIG. 4D) and flow cytometry revealed that the three-round SMNP treatments resulted in a higher HBB/GFP knockin efficiency of 21%, while showing minimum impact to cell viability and growth.

FIGS. 4A - 4D show results of optimizing the SMNP/SNSMD strategy for CRISPR/Cas9 knockin of the HBB/GFP gene in K562 3.21 cells using both Cas9•sgRNA⊂SMNPs and HBB/GFP-plasmid⊂SMNPs. FIG. 4A is a timeline depicting how different delivery time intervals (ΔT) affect CRISPR/Cas9 knockin performance. FIG. 4B are representative fluorescence images and histograms of the K562 3.21 cells obtained for ΔT = 0, 2, 4, 6, 9, or 12 h. All scale bars are 100 µm. FIG. 4C is a timeline developed for three rounds of SMNP treatments. FIG. 4D are fluorescence images and histograms of the K562 3.21 cells harvested after the three-round SMNP treatments. All scale bars are 100 µm.

The studies above prompted exploration of the feasibility of co-encapsulating both Cas9•sgRNA and HBB/GFP-plasmid into a single SMNP vector in order to simplify the complicated procedures using the two vectors. Based on the previous formulation conditions, Cas9•sgRNA+HBB/GFP-plasmid⊂SMNPs was prepared via stoichiometric mixing of the Cas9·sgRNA and HBB/GFP-plasmid with the SMNP building blocks. Also, the sizes of the co-encapsulated SMNPs via SEM and DLS was evaluated, which showed that the co-encapsulated SMNPs have larger sizes and size distributions (240±90 nm). The surface-charge density of co-encapsulated SMNPs was determined by zeta potential measurements in PBS buffer solution, which suggested that the zeta potential was 13 ± 4 mV. The resulting Cas9•sgRNA+HBB/GFP-plasmid⊂SMNPs were then used to study CRISPR/Cas9-mediated knockin, where it was found that the co-encapsulated SMNP vector exhibited significantly compromised knockin performance (5%).

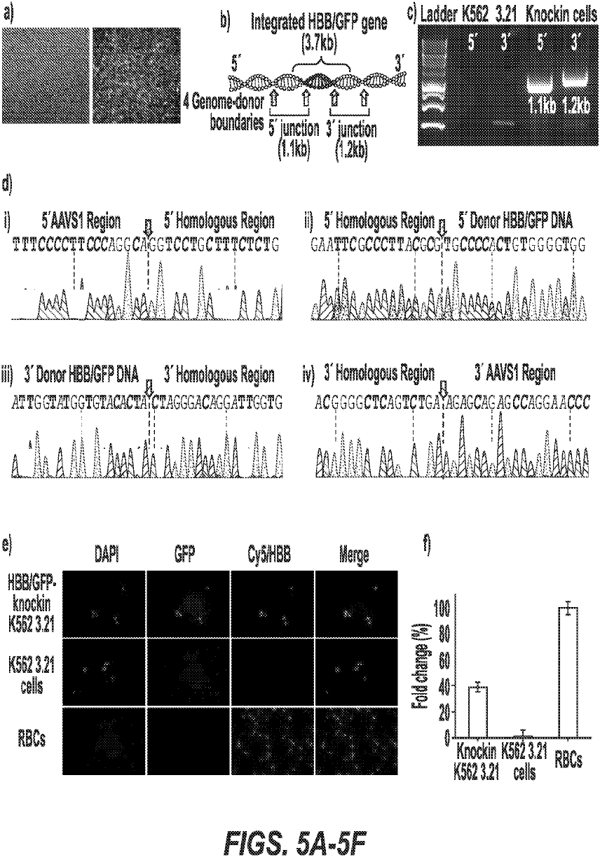

The K562 3.21 cells harvested from the three-round SMNP treatments were sorted by flow cytometry to obtain purified HBB/GFP-knockin K562 3.21 cells. Over 20 rounds of culture expansion, these cells displayed consistent GFP signals (FIG. 5A), suggesting their clonal stability. To test the CRISPR-Cas9-mediated knockin (FIG. 5B) of HBB/GFP gene into the AAVS1 site via the HDR pathway, genomic DNA from HBB/GFP-knockin K562 3.21 cells was extracted, followed by PCR analysis and Sanger sequencing. After PCR amplification, the two characteristic DNA fragments, the 5′ junction (1.1 kb) and the 3′ junction (1.2 kb) - signifying the integration of 3.7-kb HBB/GFP into the AAVS1 -were detected by electrophoresis (FIG. 5C). Sanger sequencing was employed to analyze the DNA fragments at four genome-donor boundaries (colored arrows in FIGS. 5B and 5D in both of the 5′ and 3′ junctions, indicating precise integration of the HBB/GFP gene. We further examine whether the integrated HBB gene could functionally express HBB protein. Together with two control cells (untreated K562 3.21 and red blood cells, RBCs, from a healthy donor), HBB/GFP-knockin K562 3.21 cells were subjected to IF staining. As shown in FIG. 5E, strong red fluorescence signals (marking IF-stained HBB protein) were observed in both HBB/GFP-knockin K562 3.21 and RBCs. Next, quantitative PCR was performed to quantify HBB mRNA expression (normalized against a housekeeping gene, beta actin) in these cells. The results, shown in FIG. 5F, reveal that increased HBB expression was observed for HBB/GFP-knockin K562 3.21, in contrast to the untreated K562 3.21.

FIGS. 5A-5F show results of the analysis of K562 3.21 cells for HBB/GFP gene integration into the AAVS1 site. FIG. 5A shows fluorescence microscopy images of purified HBB/GFP-knockin K562 3.21 cells after twenty rounds of culture expansion. FIG. 5B is a schematic depicting the integration of HBB/GFP gene into the AAVS1 site, in which the locations of the two homology-directed repair (HDR) junctions and four genome-donor boundaries are labeled. FIG. 5C is an electrophoretogram shows the two characteristic DNA fragments, i.e., the 5′ junction (1.1 kb) and the 3′ junction (1.2 kb). FIG. 5D shows Sanger sequencing of the four genome-donor boundaries. FIG. 5E is representative immunofluorescence images of HBB/GFP-knockin K562 3.21, K562 3.21, and RBCs. FIG. 5F shows quantitative PCR for quantification of HBB mRNA expression.

### In Vivo Study

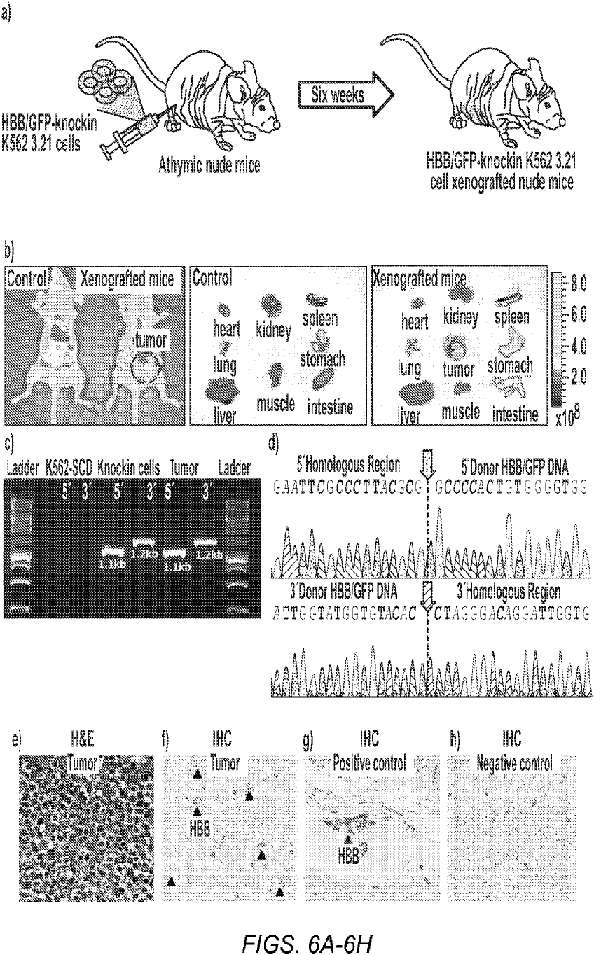

To test the proliferative potential of HBB/GFP-knockin K562 3.21 cells in vivo, approximately 1×10⁷ HBB/GFP-knockin K562 3.21 cells in 200 µL Matrigel were injected intraperitoneally (IP) in nonirradiated athymic nude mice (n=3) (FIG. 6A). Six weeks post IP injection, the mice were euthanized by cervical dislocation under deep anesthesia. To track HBB/GFP-knockin K562 3.21 cells in the xenograft tumors, a Lumina IVIS II System (Perkin Elmer, Waltham, MA) was used to image the entire peritoneal cavity, individual organs, and resected tumors. As shown in FIG. 6B, GFP signals were only observed in the xenograft tumors of the mice injected with HBB/GFP-knockin K562 3.21 cells. Subsequently, the tumor tissues were subjected to PCR analysis (FIG. 6C) and Sanger sequencing (FIG. 6D) to test the stability of the integrated HBB/GFP gene in vivo throughout the repopulating process. Tumor sections were prepared for standard pathology hematoxylin-eosin (H&E) staining and immunohistochemistry (IHC) staining for HBB. Two pathologists reviewed all slides independently, reporting: i) tumor cells were observed (FIG. 6E), and ii) HBB positivity in the cytoplasm of the tumor cells (FIG. 6F) with the RBCs being used for HBB positive control (FIG. 6G) and lymphocytes for HBB negative control (FIG. 6H). Collectively, these experimental data support that the injected HBB/GFP-knockin K562 3.21 cells maintain their ability to proliferate and maintain consistent gene expression in vivo.

FIGS. 6A-6H show the analysis of the proliferative potential of HBB/GFP-knockin K562 3.21 cells in vivo. In FIG. 6A, HBB/GFP-knockin K562 3.21 cells were introduced into athymic nude mice via intraperitoneal (IP) injection to test the cells’ in vivo growth potential. FIG. 6B shows representative fluorescence images of mice, organs, and xenograft tumors 6 weeks post IP injection. The harvested tumor tissues were subjected to (FIG. 6C) PCR analysis and (FIG. 6D) Sanger sequencing. FIGS. 6E-6H are representative images of hematoxylin-eosin H&E staining of the xenograft tumor tissue, immunohistochemistry (IHC) staining for HBB protein expression in the tumor tissue, positive control of IHC staining for HBB, and negative control of IHC staining for HBB, respectively.

### Discussion

In the context of hemoglobinopathies, two main CRISPR/Cas9 gene editing strategies have been reported, including i) knockdown of BCL11A gene via NHEJ to elevate fetal hemoglobin (HbF) levels and ii) correction of HBB gene mutations via HDR (30). Genetic variants of the BCL11A gene are found to regulate HbF expression. Knockdown of BCL11A leads to increased HbF levels in the erythroid lineage, which is being applied clinically in gene therapies for hemoglobinopathies (31). However, ubiquitous BCL11A knockdown weakened human RBC enucleation and impaired engraftment of human repopulating HSCs (5). The HDR-based strategy has been investigated for the site-specific correction of the point mutation (A-to-T) that leads to SCD at the HBB locus in human HSCs (32). However, this strategy can only cure SCD, not β-thalassemia, due to the multiple mutations associated with this disease. Moreover, failed gene correction at the HBB locus can lead to gene disruption by NHEJ, generating a β-thalassemic phenotype instead of correcting the SCD mutation (5). The AAVS1 safe-harbor locus is an ideal site in the human genome for knockin of a new gene, which does not cause alterations of the host genome and is safe for the host cell or organism (33). Hence, knockin of HBB into AAVS1 can provide a safe and general curative therapeutic solution for both SCD and β-thalassemia.

An effective CRISPR/Cas9-mediated knockin approach using a combined SMNP/SNSMD strategy is introduced here, which enables step-by-step delivery of Cas9·sgRNA complex and dDNA (HBB/GFP-plasmid) encapsulated in two different SMNP vectors. By conducting small-scale combinatorial screenings, optimal formulations were identified for the preparation of TAT-grafted Cas9•sgRNA ⊂SMNPs (average sizes = 108 ± 37 nm and 8% TAT coverage) and HBB/GFP-plasmid⊂SMNPs (average sizes = 125 ± 43 nm and 6% TAT coverage). Compared with commercially available CRISPR and plasmid delivery reagents (Lipofectamine CRISPRMax and Lipofectamine 3000), the SMNP/SNSMD strategy showed significantly higher performances in cell-uptake and GFP-transfection. Using this non-viral delivery approach, successful integration of a single-copy HBB/GFP gene into the AAVS1 safe harbor site of a SCD cell model has been demonstrated. Targeted genomic editing by CRISPR/Cas9 can efficiently generate knockout cells via the NHEJ pathway, but the efficiency of gene knockin by HDR is substantially lower (34). Furthermore, increasing the inserted gene size would reduce knockin efficiency (35). Compared with other gene knockin studies (35-40), higher knockin efficiency (21%) with longer DNA insertion (3.7-kb HBB/GFP gene) was obtained via optimization of the delivery time interval and multiple treatments of these two SMNP vectors, while keeping high cell viability. Physical contact between the cell membranes and the nanowires led to efficient uptake of SMNPs. Meanwhile, the HBB/GFP-knockin K562 3.21 cells maintained their ability to proliferate and consistent HBB gene expression in vivo, showing the feasibility of a potential therapeutic solution for hemoglobinopathies.

In summary, this proof-of-concept study highlights: i) the potential of the combined SMNP/SNSMD strategy as an effective delivery platform capable of co-delivering Cas9·sgRNA and dDNA into hematologic cells (known to be difficult to transfect), and ii) the demonstration of an efficient CRISPR/Cas9-mediated knockin of a long-frame DNA sequence (i.e., 3.7-kb HBB/GFP gene) using non-viral vectors. Further research on CD34+ hematopoietic stem cells is currently ongoing. This approach may enable CRISPR/Cas9-mediated knockin of HBB genes using autologous CD34+ cells, offering a general clinical therapeutic solution for hemoglobinopathies.

### Materials and Methods

### SgRNA Synthesis

Briefly, the sequence of sgRNA used to target the AAVS1 locus is

ggggccacuagggacaggauGUUUUAGAGCUAGAAAUAGCAAGUUAAAAU
AAGGCUAGUCCGUUAUCAACUUGAAAAAGUGGCACCGAGUCGGUGCUUUU
UU (SEQ ID NO:8),

which was synthesized by Integrated DNA Technologies, Inc. (IDT, Iowa).

### Cell Culture

The human leukemia K562 3.21 cells were cultured in a humidified atmosphere of 5% CO₂/air in RPMI-1640 medium, supplemented with 10% fetal bovine serum, 100 units/ml penicillin and 100 µg/ml streptomycin. Individual wells of an 8-well plate were inoculated with complete medium containing 10,000 of K562 3.21 cells per milliliter. The plates were incubated at 37° C. in a humidified 5% incubator for 18 h prior to the experiments.

### Synthesis of EGFP-Cas9•sgRNA⊂SMNPs

Self-assembly was used to prepare the EGFP-Cas9 protein and sgRNA encapsulated supramolecular nanoparticles (EGFP-Cas9•sgRNA⊂SMNPs). Three batches of EGFP-Cas9•sgRNA⊂SMNPs were formulated by systemically modulating i) the weight ratios (wt%) between SMNP vector and EGFP-Cas9•sgRNA payload, ii) sizes of EGFP-Cas9•sgRNA⊂SMNPs, and iii) the percentages of TAT ligand on SMNP surfaces. The optimized formulation was prepared as follows. A total of 2.0 µL DMSO solution containing Ad-PAMAM (7.2 µg) was added into a 50 µL PBS mixture with EGFP-Cas9 protein (3.0 µg), sgRNA (0.6 µg, Mol ratio≈1:1), Ad-PEG (45 µg), CD-PEI (20 µg), and Ad-PEG-TAT (3.6 µg). The resulting mixture was then stirred vigorously to achieve optimal Cas9•sgRNA⊂SNMPs. The mixture was stored at 4° C. for 1 h; afterward, DLS, SEM, and TEM were used to characterize the sizes of EGFP-Cas9•sgRNA⊂SMNPs.

### Delivery EGFP-Cas9•sgRNA⊂SMNPs to K562 3.21 Cells

Prior to settling the cells onto Ad-SiMWS, K562 3.21 cells were first starved in serum-free RPMI medium in 75 cm² cell culture flask for 10 h to synchronize cells to G0/G1 phases of cell cycle (29), then centrifuged to remove medium at the rate of 300 g for 5 min. Approximately K562 3.21 cells (1×10⁵ in 1.0 mL serum-containing RPMI medium) were introduced into each well of an 8-well plate, where a 2.5×3 cm² Ad-SiNWS was placed. Finally, the Cas9•sgRNA⊂SNMPs (containing 3.0 µg Cas9 protein) in 1.0 mL serum-containing RPMI medium was added to each well. The cells were co-incubated with SMNPs for a certain period. Every 48 h, 1.0 mL medium was removed via pipette and then new 1.0 mL serum-containing RPMI medium was added to each well. After washing with PBS, the cells in the well were immediately fixed with 2% PFA and then stained with DAPI. Microscopy-based image cytometry was used to detect the cellular uptake performances of different formulations. After each treatment, the GFP signal was quantified with a fluorescent microscope equipped with a CCD camera (Nikon TE2000S, Japan).

### Delivery Cas9•sgRNA⊂Cy5-SMNPs to K562 3.21 Cells

The growth-synchronized K562 3.21 cells (1×10⁵ in 1.0 mL serum-containing RPMI medium) were introduced into each well of an 8-well plate, where a 2.5×3 cm² Ad-SiNWS was placed. The Cas9•sgRNA⊂Cy5-SMNPs (containing 3.0 µg Cas9 protein) in 1.0 mL serum-containing RPMI medium was added to each well. The cells were co-incubated with SMNPs for a certain period. Every 48 h, 1.0 mL medium was removed from each well and replaced with 1.0 mL serum-containing RPMI medium. After washing with PBS, the cells in the well were immediately fixed with 2% PFA, and then stained with DAPI. Microscopy-based image cytometry was used to detect the cellular uptake performances of different formulations. After different treatments, the Cy5 signal was quantified using a fluorescent microscope with a CCD camera (Nikon TE2000S, Japan).

### Off-Target Analysis

Off-target analysis of sgRNA-AAVS1 was performed using the Cas-OFFinder design tool (http://www.rgenome.net/cas-offinder/). The top 5 off-target hits with canonical PAM sequence based on the query were:

GGGGCaACTAGaGACAGGAaGGG, (chromosome 8, 3 mismatc
hes) (SEQID NO:9),

GGtGCCACTAGGcACAGGAgCGG, (chromosome 8, 3 mismatc
hes) (SEQID NO:10),

tGGGCCACTAtGGACAGGAaTGG, (chromosome 12, 3 mismat
ches) (SEQID NO: 11),

GaGGCCACcAGGGACAGGcTGGG (chromosome 5, 3 mismatch
es) (SEQID NO: 12),

GGGGtacCTAGGGtCAGGATGGG (chromosome 8, 4 mismatch
es) (SEQ IDNO: 13).

The PCR primers for these regions were designed accordingly.

### Synthesis of HBB/GFP-Plasmid⊂SMNPs

A similar self-assembly procedure was applied to prepare the HBB/GFP-plasmid⊂SMNPs. Three batches of HBB/GFP-plasmid⊂SMNPs were formulated by systematically modulating i) the weight ratios (wt%) between SMNP vector and EGFP-Cas9·sgRNA payload, ii) the sizes of HBB/GFP-plasmid⊂SMNPs, and iii) the percentages of TAT ligand on SMNP surfaces. The optimized formulation was synthesized as follows. A total of 2.0 µL DMSO solution containing Ad-PAMAM (15 µg) was added into a 50 µL PBS mixture with HBB/GFP-plasmid (1.0 µg), Ad-PEG (23 µg), CD-PEI (10 µg), and Ad-PEG-TAT (1.4 µg). The resulting mixture was then stirred vigorously to generate optimal HBB/GFP-plasmid⊂SMNPs.

### Delivery HBB/GFP-Plasmid⊂SMNPs to K562 3.21 Cells

The growth-synchronized K562 3.21 cells (1×10⁵ in 1.0 mL serum-containing RPMI medium) were introduced into each well of an 8-well plate, where a 2.5×3 cm² Ad-SiNWS was placed. The HBB/GFP-plasmid⊂SMNPs (containing 2.0 µg HBB/GFP-plasmid) in 1.0 mL serum-containing RPMI medium was added to the well. The cells were co-incubated with SMNPs for a certain period. After washing with PBS, the cells in each chamber were immediately fixed with 2% PFA, and then stained with DAPI. Microscopy-based image cytometry was used to detect the cellular uptake performances of different formulations.

### Delivery Cas9•sgRNA⊂SMNPs and HBB/GFP-Plasmid⊂SMNPs to K562 3.21 Cells for Gene Knockin Study

The growth-synchronized K562 3.21 cells (1 × 10⁵ in 1.0 mL serum-containing RPMI medium) were introduced into each well of an 8-well plate. First was examined how delivery time interval (ΔT = 0, 2, 4, 6, 9, or 12 h) between Cas9•sgRNA⊂SMNPs (containing 3.0 µg Cas9 protein in 0.5 mL serum-containing RPMI medium) and HBB/GFP-plasmid⊂SMNPs (containing 2.0 µg HBB/GFP-plasmid in 0.5 mL serum-containing RPMI medium) affected the knockin efficiency in K562 3.21 cells. The cells were co-incubated with two SMNPs for 10 days. Every 2 days, 1.0 mL medium was removed using a pipette and then new 1.0 mL serum containing RPMI medium was added to each well. After washing with PBS, the cells in the well were immediately fixed with 2% PFA and then stained with DAPI. Microscopy-based image cytometry was used to measure the delivery performances of different conditions.

### Delivery Cas9•sgRNA+HBB/GFP-Plasmid⊂SMNPs to K562 3.21 Cells for Gene Knockin Study

The growth-synchronized K562 3.21 cells (1 × 10⁵ in 1.0 mL serum-containing RPMI medium) were introduced into each well of an 8-well plate. The Cas9•sgRNA+HBB/GFP-plasmid⊂SMNPs (containing 3.0 µg Cas9 protein and 2.0 µg HBB/GFP-plasmid in 1.0 mL serum-containing RPMI medium) was added to each well. The cells were co-incubated with SMNPs for 10 days. Every 2 days, 1.0 mL medium was removed using a pipette and then 1.0 mL new serum containing RPMI medium was added to each well. After washing with PBS, the cells in the well were immediately fixed with 2% PFA, stained with DAPI, and then evaluated via microscopy-based image cytometry to evaluate the delivery performance of different conditions.

### DNA Extraction and PCR

The HBB/GFP-knockin K562 3.21 cells were harvested and then washed with PBS. The genomic DNA was extracted with a commercial QIAamp® DNA Mini Kit (Qiagen, Germany) using the manufacturer’s instructions. Then, PCR was conducted to amplify integrated HBB/GFP gene with a S1000TM Thermal Cycler (Bio-Rad) under the following PCR conditions: 95° C. for 3 min followed by 35 cycles (95° C. for 15 s, 58° C. for 15 s and 72° C. for 20 s) and 72° C. for 3 min. The PCR products were checked on a 1.5% electrophoresis gel.

The PCR primer sequences are listed as follows:

5′ Junction forward primer: 5′-CCGGAACTCTGCCCTCTA
AC-3′ (SEQ IDNO:14),

5′ Junction reverse primer: 5′-AGTAGGAAAGTCCCATAA
GGTCA-3′ (SEQID NO: 15);

3′ Junction forward Primer: 5′-AAGCTCATCTGGTCTCCC
TTCC-3′ (SEQID NO: 16),

3′ Junction reverse Primer: 5′-TCCTGGGATACCCCGAAG
AG-3′ (SEQ IDNO: 17).

### Quantitative PCR

After adding TRIzol (800 µl), the cells were homogenized, treated with chloroform (160 µl) and centrifuged for 15 min at 4° C. The aqueous phase of the sample was removed by pipette and 100% isopropanol (400 µl) was added. After being centrifuged for 10 min, the supernatant was removed from the tube, and the pellet was washed with 75% ethanol and centrifuged for 5 min. Afterwards, the supernatant was removed, and the pellet was dissolved in DNase- and RNase-free water. RNA (1 µg) was reverse transcribed using the SuperScript III First-Strand Synthesis kit. qPCR analysis was performed using PowerUp SYBR Green Master Mix (Applied Biosystems, California) with the primers below. Values were normalized against the gene expression of the housekeeping gene beta-actin.

qPCR primer beta-globin forward primer: 5′-CTCATG
GCAAGAAAGTGCTCG-3′ (SEQ ID NO: 18)

Reverse Primer: 5′-AATTCTTTGCCAAAGTGATGGG-3′ (SEQ
ID NO: 19)

### Immunofluorescence Staining

The living cells were fixed in 4% paraformaldehyde, permeabilized in 0.1% Triton X-100, and blocked in 5% normal bovine serum albumin (BSA) in PBS. The cells were incubated with HBB antibody (Abcam ab214049). After being washed three times with PBS, the cells were incubated with secondary antibodies (Donkey anti Rabbit) conjugated with Cy5 (red). DAPI (blue) was used as the nuclear stain. Labeled cells were imaged with a laser-scanning confocal microscope (Olympus). The total amount of retained immunofluorescent material was determined in the green (488 nm) and the red (546 nm) channels.

### In Vivo Studies

All animal studies were conducted under an approved Institutional Animal Care and Use Committee protocol. A total of 1×10⁷ HBB/GFP-knockin K562 3.21 cells in 200 µl Matrigel were injected intraperitoneally (IP) in nonirradiated athymic nude female mice (n=3, 6-7 weeks of age, Envigo company) under brief isoflurane anesthesia. Tumors were staged for 6 weeks. Then, the animals were euthanized and their peritoneal cavities were exposed. For the visualization of HBB/GFP-knockin K562 3.21 cells in organs and tumor, the organs were excised from the peritoneal cavities and placed on a plate with a white background. The Lumina IVIS II System (Perkin Elmer, Waltham, MA) was used to obtain fluorescent images of the whole peritoneal cavity, individual organs, and tumors. Living Image Software version 4.1 (Perkin Elmer) was used for GFP fluorescence analysis. To aid in visualization, the GFP signal was pseudocolored by applying a logarithmic grayscale to the whole peritoneal cavity image or by applying reverse logarithmic grayscale to the images of the organs and tumor. Genomic DNA was extracted from the tumor of each mouse by the Dneasy blood and Tissue Kit (Qiagen GmbH, Germany). The presence of human-specific DNA within the transplanted mice was confirmed by PCR and Sanger sequencing. The immunohistochemistry experiments were carried out on 4 µm thick formalin-fixed and paraffin-embedded tissue sections. The sections were prepared for standard pathology hematoxylin-eosin (H&E) staining and immunohistochemistry (IHC) staining for HBB using a commercially available detection kit (Dako EnVision Plus-HRP, Dako), according to the manufacturer’s instructions.

### REFERENCES

1. T. N. Williams, D. J. Weatherall, World distribution, population genetics, and health burden of the hemoglobinopathies. Cold Spring Harb. Perspect. Med. 2, a011692 (2012).

2. M. M. Sirdah, J. Sievertsen, M. S. Al-Yazji, I. S. Tarazi, R. M. Al-Haddad, R. D. Horstmann, C. Timmann, The spectrum of β-thalassemia mutations in Gaza Strip, Palestine. Blood Cells Mol. Dis. 50, 247-251 (2013).

3. V. Compernolle, S. T. Chou, S. Tanael, W. Savage, J. Howard, C. D. Josephson, I. Odame, C. Hogan, G. Denomme, N. Shehata, Red blood cell specifications for patients with hemoglobinopathies: a systematic review and guideline. Transfusion 58, 1555-1566 (2018).

4. A. King, S. Shenoy, Evidence-based focused review of the status of hematopoietic stem cell transplantation as treatment of sickle cell disease and thalassemia. Blood 123, 3089-3094 (2014).

5. E. Magrin, A. Miccio, M. Cavazzana, Lentiviral and genome-editing strategies for the treatment of β-hemoglobinopathies. Blood 134, 1203-1213 (2019).

6. M. Cavazzana, C. Antoniani, A. Miccio, Gene therapy for β-hemoglobinopathies. Mol. Ther. 25, 1142-1154 (2017).

7. J. A. Doudna, E. Charpentier, The new frontier of genome engineering with CRISPR-Cas9. Science 346, 1258096 (2014).

8. J. A. Doudna, The promise and challenge of therapeutic genome editing. Nature 578, 229-236 (2020).

9. L. Li, S. Hu, X. Chen, Non-viral delivery systems for CRISPR/Cas9-based genome editing: Challenges and opportunities. Biomaterials 171, 207-218 (2018).

10. E. Senis, C. Fatouros, S. GroBe, E. Wiedtke, D. Niopek, A. K. Mueller, K. Bömer, D. Grimm, CRISPR/Cas9-mediated genome engineering: an adeno-associated viral (AAV) vector toolbox. Biotechnol. J. 9, 1402-1412 (2014).

11. Z. Wu, H. Yang, P. Colosi, Effect of genome size on AAV vector packaging. Mol. Ther. 18, 80-86 (2010).

12. H.-X. Wang, M. Li, C. M. Lee, S. Chakraborty, H.-W. Kim, G. Bao, K. W. Leong, CRISPR/Cas9-based genome editing for disease modeling and therapy: challenges and opportunities for nonviral delivery. Chem. Rev. 117, 9874-9906 (2017).

13. Y. Rui, D. R. Wilson, J. J. Green, Non-viral delivery to enable genome editing. Trends Biotechnol. 37, 281-293 (2019).

14. L. Li, Z. Yang, S. Zhu, L. He, W. Fan, W. Tang, J. Zou, Z. Shen, M. Zhang, L. Tang, A rationally designed semiconducting polymer brush for NIR-II imaging-guided light-triggered remote control of CRISPR/Cas9 genome editing. Adv. Mater. 31, 1901187 (2019).

15. J. Liu, J. Chang, Y. Jiang, X. Meng, T. Sun, L. Mao, Q. Xu, M. Wang, Fast and efficient CRISPR/Cas9 genome editing in vivo enabled by bioreducible lipid and messenger RNA nanoparticles. Adv. Mater., 1902575 (2019).

16. Y. Rui, D. R. Wilson, J. Choi, M. Varanasi, K. Sanders, J. Karlsson, M. Lim, J. J. Green, Carboxylated branched poly (β-amino ester) nanoparticles enable robust cytosolic protein delivery and CRISPR-Cas9 gene editing. Sci. Adv. 5, eaay3255 (2019).

17. K. Lee, M. Conboy, H. M. Park, F. Jiang, H. J. Kim, M. A. Dewitt, V. A. Mackley, K. Chang, A. Rao, C. Skinner, Nanoparticle delivery of Cas9 ribonucleoprotein and donor DNA in vivo induces homology-directed DNA repair. Nat. Biomed. Eng. 1, 889-901 (2017).

18. Q. Cheng, T. Wei, L. Farbiak, L. T. Johnson, S. A. Dilliard, D. J. Siegwart, Selective organ targeting (SORT) nanoparticles for tissue-specific mRNA delivery and CRISPR-Cas gene editing. Nat. Nanotech. 15, 313-320 (2020).

19. B. Lee, K. Lee, S. Panda, R. Gonzales-Rojas, A. Chong, V. Bugay, H. M. Park, R. Brenner, N. Murthy, H. Y. Lee, Nanoparticle delivery of CRISPR into the brain rescues a mouse model of fragile X syndrome from exaggerated repetitive behaviours. Nat. Biomed. Eng. 2, 497 (2018).

20. P. Mora-Raimundo, D. Lozano, M. Manzano, M. Vallet-Regi, Nanoparticles to knockdown osteoporosis-related gene and promote osteogenic markers expression for osteoporosis treatment. ACS Nano 13, 5451-5464 (2019).

21. K. Lee, M. Conboy, H. M. Park, F. Jiang, H. J. Kim, M. A. Dewitt, V. A. Mackley, K. Chang, A. Rao, C. Skinner, Nanoparticle delivery of Cas9 ribonucleoprotein and donor DNA in vivo induces homology-directed DNA repair. Nat. Biomed. Eng. 1, 889 (2017).

22. H.-X. Wang, Z. Song, Y.-H. Lao, X. Xu, J. Gong, D. Cheng, S. Chakraborty, J. S. Park, M. Li, D. Huang, Nonviral gene editing via CRISPR/Cas9 delivery by membrane-disruptive and endosomolytic helical polypeptide. Proc. Natl. Acad. Sci. U. S. A. 115, 4903-4908 (2018).

23. H. Wang, S. Wang, H. Su, K. J. Chen, A. L. Armijo, W. Y. Lin, Y. Wang, J. Sun, K. i. Kamei, J. Czemin, A supramolecular approach for preparation of size-controlled nanoparticles. Angew. Chem. Int. Ed. 48, 4344-4348 (2009).

24. F. Wang, P. Yang, J.-s. Choi, P. Antovski, Y. Zhu, X. Xu, T.-H. Kuo, L.-E. Lin, D. N. Kim, P.-C. Huang, Cross-linked fluorescent supramolecular nanoparticles for intradermal controlled release of antifungal drug—A therapeutic approach for onychomycosis. ACS Nano 12, 6851-6859 (2018).

25. J. Peng, M. A. Garcia, J.-s. Choi, L. Zhao, K.-J. Chen, J. R. Bernstein, P. Peyda, Y.-S. Hsiao, K. W. Liu, W.-Y. Lin, Molecular recognition enables nanosubstrate-mediated delivery of gene-encapsulated nanoparticles with high efficiency. ACS Nano 8, 4621-4629 (2014).

26. M. D. Hoban, D. Lumaquin, C. Y. Kuo, Z. Romero, J. Long, M. Ho, C. S. Young, M. Mojadidi, S. Fitz-Gibbon, A. R. Cooper, CRISPR/Cas9-mediated correction of the sickle mutation in human CD34+ cells. Mol. Ther. 24, 1561-1569 (2016).

27. Y. Liu, J. Du, J. s. Choi, K. J. Chen, S. Hou, M. Yan, W. Y. Lin, K. S. Chen, T. Ro, G. S. Lipshutz, A High-Throughput Platform for Formulating and Screening Multifunctional Nanoparticles Capable of Simultaneous Delivery of Genes and Transcription Factors. Angew. Chem. Int. Ed. 55, 169-173 (2016).

28. V. T. Chu, T. Weber, B. Wefers, W. Wurst, S. Sander, K. Rajewsky, R. Kühn, Increasing the efficiency of homology-directed repair for CRISPR-Cas9-induced precise gene editing in mammalian cells. Nat. Biotechnol. 33, 543 (2015).

29. S. E. Golding, E. Rosenberg, A. Khalil, A. McEwen, M. Holmes, S. Neill, L. F. Povirk, K. Valerie, Double strand break repair by homologous recombination is regulated by cell cycle-independent signaling via ATM in human glioma cells. J. Biol. Chem. 279, 15402-15410 (2004).

30. M. R. Lidonnici, G. Ferrari, Gene therapy and gene editing strategies for hemoglobinopathies. Blood Cells Mol. Dis. 70, 87-101 (2018).

31. M. C. Canver, E. C. Smith, F. Sher, L. Pinello, N. E. Sanjana, O. Shalem, D. D. Chen, P. G. Schupp, D. S. Vinjamur, S. P. Garcia, BCL11A enhancer dissection by Cas9-mediated in situ saturating mutagenesis. Nature 527, 192 (2015).

32. D. P. Dever, R. O. Bak, A. Reinisch, J. Camarena, G. Washington, C. E. Nicolas, M. Pavel-Dinu, N. Saxena, A. B. Wilkens, S. Mantri, CRISPR/Cas9 β-globin gene targeting in human haematopoietic stem cells. Nature 539, 384 (2016).

33. E. P. Papapetrou, A. Schambach, Gene insertion into genomic safe harbors for human gene therapy. Mol. Ther. 24, 678-684 (2016).

34. C. D. Richardson, G. J. Ray, M. A. DeWitt, G. L. Curie, J. E. Corn, Enhancing homology-directed genome editing by catalytically active and inactive CRISPR-Cas9 using asymmetric donor DNA. Nat. Biotechnol. 34, 339-344 (2016).

35. A. Paix, A. Folkmann, D. H. Goldman, H. Kulaga, M. J. Grzelak, D. Rasoloson, S. Paidemarry, R. Green, R. R. Reed, G. Seydoux, Precision genome editing using synthesis-dependent repair of Cas9-induced DNA breaks. Proc. Natl. Acad. Sci. U. S. A. 114, E10745-E10754 (2017).

36. J.-P. Zhang, X.-L. Li, G.-H. Li, W. Chen, C. Arakaki, G. D. Botimer, D. Baylink, L. Zhang, W. Wen, Y.-W. Fu, Efficient precise knockin with a double cut HDR donor after CRISPR/Cas9-mediated double-stranded DNA cleavage. Genome Biol. 18, 35 (2017).

37. X. Yao, X. Wang, J. Liu, X. Hu, L. Shi, X. Shen, W. Ying, X. Sun, X. Wang, P. Huang, CRISPR/Cas9-Mediated precise targeted integration in vivo using a double cut donor with short homology arms. EBioMedicine 20, 19-26 (2017).

38. X. Yao, X. Wang, X. Hu, Z. Liu, J. Liu, H. Zhou, X. Shen, Y. Wei, Z. Huang, W. Ying, Homology-mediated end joining-based targeted integration using CRISPR/Cas9. Cell Res. 27, 801-814 (2017).

39. J. Song, D. Yang, J. Xu, T. Zhu, Y. E. Chen, J. Zhang, RS-1 enhances CRISPR/Cas9-and TALEN-mediated knock-in efficiency. Nat. Commun. 7, 1-7 (2016).

40. X. He, C. Tan, F. Wang, Y. Wang, R. Zhou, D. Cui, W. You, H. Zhao, J. Ren, B. Feng, Knock-in of large reporter genes in human cells via CRISPR/Cas9-induced homology-dependent and independent DNA repair. Nucleic Acids Res. 44, e85-e85 (2016).

We claim:

1. A composition for delivering an endonuclease and a nucleic acid sequence encoding a donor protein to a cell, comprising:
a first plurality of self-assembled supramolecular nanoparticles (SMNPs), each of the first plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex, wherein the plurality of binding components and the plurality of cores self-assemble when brought into contact to form the first plurality of self-assembled supramolecular nanoparticles (SMNPs);
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle;
the endonuclease; and
a nucleotide sequence comprising a recognition sequence specific to the endonuclease; and
a second plurality of self-assembled supramolecular nanoparticles (SMNPs), each of the second plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex, wherein the plurality of binding components and the plurality of cores self-assemble when brought into contact to form the second plurality of self-assembled supramolecular nanoparticles (SMNPs);
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and
the nucleic acid sequence encoding the donor protein,
wherein the endonuclease and the nucleotide sequence comprising a recognition sequence specific to the endonuclease are encapsulated within each of the first plurality of self-assembled SMNPs, and
wherein the nucleic acid sequence encoding the donor protein is encapsulated within each of the second plurality of self-assembled SMNPs.

2. The composition of claim 1, wherein the endonuclease is a CRISPR associated protein 9 (Cas9), and
wherein the nucleotide sequence is a single guide RNA (sgRNA).

3. The composition of claim 1, wherein the first plurality of self-assembled SMNPs and the endonuclease are present in a percent mass (w/w) ratio of between 100:1 to 100:50.

4. The composition of claim 1, wherein the second plurality of self-assembled SMNPs and the nucleic acid sequence encoding the donor protein are present in a percent mass (w/w) ratio of between 100:1 and 100:50.

5. The composition of claim 1, wherein each of the first plurality of self-assembled SMNPs and each of the second plurality of self-assembled SMNPs have a diameter of between 100 nanometers and 200 nanometers.

6. The composition of claim 1, wherein the first plurality of self-assembled SMNPs and the second plurality of SMNPs further comprise a membrane penetration ligand coupled to an outer surface.

7. The composition of claim 1, wherein the plurality of binding components of the first and second plurality of self-assembled SMNPs comprises polythylenimine, poly(L-lysine), or poly(β-amino ester).

8. The composition of claim 1, wherein the plurality of binding regions of the first and second plurality of self-assembled SMNPs comprises beta-cyclodextrin, alpha-cyclodextrin, gamma-cyclodextrin, cucurbituril or calixarene.

9. The composition of claim 1, wherein the plurality of cores of the first and second plurality of self-assembled SMNPs comprises polyamidoamine dendrimers, poly(prophylenimine) (PPI) dendrimer, triazine dendrimer, carbosilane dendrimer, poly(ether imine) (PETIM) dendrimer or phosphorus dendrimer.

10. The composition of claim 1, wherein the at least one core binding element of the first and second plurality of self-assembled SMNPs comprises adamantane, azobenzene, ferrocene or anthracene.

11. The composition of claim 1, wherein the plurality of terminating components of the first and second plurality of self-assembled SMNPs comprises polyethylene glycol (PEG) or poly(propylene glycol) (PGG).

12. The composition of claim 1, wherein the single terminating binding element of the first and second plurality of self-assembled SMNPs comprises adamantane, azobenzene, ferrocene or anthracene.

13. A system for delivering an endonuclease and a nucleic acid sequence encoding a donor protein to a cell comprising:
a first plurality of self-assembled supramolecular nanoparticles (SMNPs), each of the first plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex, wherein the plurality of binding components and the plurality of cores self-assemble when brought into contact to form the first plurality of self-assembled supramolecular nanoparticles (SMNPs);
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle;
the endonuclease; and
a nucleotide sequence comprising a recognition sequence specific to the endonuclease;
a second plurality of self-assembled supramolecular nanoparticles (SMNPs), each of the second plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex, wherein the plurality of binding components and the plurality of cores self-assemble when brought into contact to form the second plurality of self-assembled supramolecular nanoparticles (SMNPs);
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and
the nucleic acid sequence encoding the donor protein; and
a device for capturing the cell, the device comprising:
a substrate; and
a plurality of nanowires at least one of attached to or integral with a surface of said substrate such that each nanowire of said plurality of nanowires has an unattached end,
wherein the endonuclease and the nucleotide sequence comprising a recognition sequence specific to the endonuclease are encapsulated within each of the first plurality of self-assembled SMNPs, and
wherein the nucleic acid sequence encoding the donor protein is encapsulated within each of the second plurality of self-assembled SMNPs.

14. The system of claim 13, wherein at least one of the first plurality of self-assembled SMNPs and the second plurality of self-assembled SMNPs are configures to reversibly attach to the plurality of nanowires.

15. The system of claim 13, wherein the endonuclease is a CRISPR associated protein 9 (Cas9), and
wherein the nucleotide sequence is a single guide RNA (sgRNA).

16. The system of claim 13, wherein the first plurality of self-assembled SMNPs and the endonuclease are present in a percent mass (w/w) ratio of between 100:1 to 100:50.

17. The system of claim 13, wherein the second plurality of self-assembled SMNPs and the nucleic acid sequence encoding the donor protein are present in a percent mass (w/w) ratio of between 100:1 and 100:50.

18. The system of claim 13, wherein each of the first plurality of self-assembled SMNPs and each of the second plurality of self-assembled SMNPs have a diameter of between 100 nanometers and 200 nanometers.

19. The system of claim 13, wherein the first plurality of self-assembled SMNPs and the second plurality of SMNPs further comprise a membrane penetration ligand coupled to an outer surface.

20. The system of claim 13, wherein the plurality of binding components of the first and second plurality of self-assembled SMNPs comprises polythylenimine, poly(L-lysine), or poly(β-amino ester).

21. The system of claim 13, wherein the plurality of binding regions of the first and second plurality of self-assembled SMNPs comprises beta-cyclodextrin, alpha-cyclodextrin, gamma-cyclodextrin, cucurbituril or calixarene.

22. The system of claim 13, wherein the plurality of cores of the first and second plurality of self-assembled SMNPs comprises polyamidoamine dendrimers, poly(prophylenimine) (PPI) dendrimer, triazine dendrimer, carbosilane dendrimer, poly(ether imine) (PETIM) dendrimer or phosphorus dendrimer.

23. The system of claim 13, wherein the at least one core binding element of the first and second plurality of self-assembled SMNPs comprises adamantane, azobenzene, ferrocene or anthracene.

24. The system of claim 13, wherein the plurality of terminating components of the first and second plurality of self-assembled SMNPs comprises polyethylene glycol (PEG) or poly(propylene glycol) (PGG).

25. The system of claim 13, wherein the single terminating binding element of the first and second plurality of self-assembled SMNPs comprises adamantane, azobenzene, ferrocene or anthracene.

26. The system of claim 13, wherein the plurality of nanowires has a diameter of between 50 nanometers and 100 nanometers.

27. The system of claim 13, wherein the plurality of nanowires has a length of between 5 micrometers and 10 micrometers.

28. The system of claim 13, wherein the plurality of nanowires comprises silicon, gold, silver, Si02, or Ti02.

29. A method for delivering an endonuclease and a nucleic acid sequence encoding a donor protein to a cell comprising:
providing a first plurality of self-assembled supramolecular nanoparticles (SMNPs);
providing a second plurality of self-assembled SMNPs;
contacting the cell with at least one of the first plurality of self-assembled SMNPs, such that the at least one of the first plurality of self-assembled SMNPs is taken up by the cell; and
contacting the cell with at least one of the second plurality of self-assembled SMNPs, such that the at least one of the second plurality of self-assembled SMNPs is taken up by the cell,
wherein each of the first plurality of self-assembled supramolecular nanoparticles SMNPs comprises:
a plurality of binding components, each having a plurality of binding regions;
a plurality of cores that are suitable to at least provide some mechanical structure to the plurality of self-assembled SMNPs, the plurality of cores comprising at least one core binding element adapted to bind to the binding regions to form a first inclusion complex, wherein the plurality of binding components and the plurality of cores self-assemble when brought into contact to form the first plurality of self-assembled SMNPs;
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle;
the endonuclease; and
a nucleotide sequence comprising a recognition sequence specific to the endonuclease,
wherein each of the second plurality of self-assembled SMNPs comprises:
a plurality of binding components, each having a plurality of binding regions;
a plurality of cores that are suitable to at least provide some mechanical structure to the plurality of self-assembled SMNPs, the plurality of cores comprising at least one core binding element adapted to bind to the binding regions to form a first inclusion complex, wherein the plurality of binding components and the plurality of cores self-assemble when brought into contact to form the second plurality of self-assembled SMNPs;
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and
the nucleic acid sequence encoding the donor protein,
wherein the nucleic acid sequence encoding the endonuclease and the nucleotide sequence comprising a recognition sequence specific to the endonuclease are encapsulated within each of the first plurality of self-assembled SMNPs, and
wherein the nucleic acid sequence encoding the donor protein is encapsulated within each of the second plurality of self-assembled SMNPs.

30. The method of claim 29, wherein the endonuclease is a CRISPR associated protein 9 (Cas9), and
wherein the nucleotide sequence is a single guide RNA (sgRNA).

31. The method of claim 29, wherein said contacting the cell with at least one of the second plurality of self-assembled SMNPs is carried out between 1 to 12 hours after said contacting the cell with at least one of the first plurality of self-assembled SMNPs.

32. The method of claim 29, wherein said contacting the cell with at least one of the second plurality of self-assembled SMNPs is carried out between 1 to 12 hours after said contacting the cell with at least one of the first plurality of self-assembled SMNPs is repeated.

33. The method of claim 29, further comprising contacting the cell with a device for capturing the cell prior to said contacting the cell with at least one of the first plurality of self-assembled SMNPs,
wherein the device for capturing the cell, the device comprises:
a substrate; and
a plurality of nanowires at least one of attached to or integral with a surface of said substrate such that each nanowire of said plurality of nanowires has an unattached end.

34. The method of claim 29, wherein the first plurality of self-assembled SMNPs and the endonuclease are present in a percent mass (w/w) ratio of between 100:1 to 100:50.

35. The method of claim 29, wherein the second plurality of self-assembled SMNPs and the nucleic acid sequence encoding the donor protein are present in a percent mass (w/w) ratio of between 100:1 and 100:50.

36. The method of claim 29, wherein each of the first plurality of self-assembled SMNPs and each of the second plurality of self-assembled SMNPs have a diameter of between 100 nanometers and 200 nanometers.

37. The method of claim 29, wherein the first plurality of self-assembled SMNPs and the second plurality of SMNPs further comprise a membrane penetration ligand coupled to an outer surface.

38. The method of claim 29, wherein the plurality of binding components of the first and second plurality of self-assembled SMNPs comprises polythylenimine, poly(L-lysine), or poly(β-amino ester).

39. The method of claim 29, wherein the plurality of binding regions of the first and second plurality of self-assembled SMNPs comprises beta-cyclodextrin, alpha-cyclodextrin, gamma-cyclodextrin, cucurbituril or calixarene.

40. The method of claim 29, wherein the plurality of cores of the first and second plurality of self-assembled SMNPs comprises polyamidoamine dendrimers, poly(prophylenimine) (PPI) dendrimer, triazine dendrimer, carbosilane dendrimer, poly(ether imine) (PETIM) dendrimer or phosphorus dendrimer.

41. The method of claim 29, wherein the at least one core binding element of the first and second plurality of self-assembled SMNPs comprises adamantane, azobenzene, ferrocene or anthracene.

42. The method of claim 29, wherein the plurality of terminating components of the first and second plurality of self-assembled SMNPs comprises polyethylene glycol (PEG) or poly(propylene glycol) (PGG).

43. The method of claim 29, wherein the single terminating binding element of the first and second plurality of self-assembled SMNPs comprises adamantane, azobenzene, ferrocene or anthracene.


SEQUENCE LISTING (19 sequences)

SEQ ID NO: 1 - protein, 12 aa, Artificial Sequence
  Description of Artificial Sequence: Synthetic peptide
GRKKRRQRRRPQ

SEQ ID NO: 2 - protein, 9 aa, Artificial Sequence
  Description of Artificial Sequence: Synthetic peptide
CRGDKGPDC

SEQ ID NO: 3 - protein, 27 aa, Artificial Sequence
  Description of Artificial Sequence: Synthetic peptide
GLAFLGFLGAAGSTMGAWSQPKKKRKV

SEQ ID NO: 4 - protein, 20 aa, Artificial Sequence
  Description of Artificial Sequence: Synthetic peptide
KETWWETWWTEWSQPKKRKV

SEQ ID NO: 5 - protein, 30 aa, Artificial Sequence
  Description of Artificial Sequence: Synthetic polypeptide
WEAALAEALAEALAEHLAEALAEALEALAA

SEQ ID NO: 6 - protein, 18 aa, Artificial Sequence
  Description of Artificial Sequence: Synthetic peptide
QLALQLALQALQAALQLA

SEQ ID NO: 7 - protein, 18 aa, Artificial Sequence
  Description of Artificial Sequence: Synthetic peptide
LKTLTETLKELTKTLTEL

SEQ ID NO: 8 - RNA, 102 nt, Artificial Sequence
  Description of Artificial Sequence: Synthetic polynucleotide
ggggccacuagggacaggauguuuuagagcuagaaauagcaaguuaaaauaaggcuaguccguuaucaacuugaaaaaguggcaccgagucggugcuuuuuu

SEQ ID NO: 9 - DNA, 23 bp, Artificial Sequence
  Description of Artificial Sequence: Synthetic oligonucleotide
ggggcaactagagacaggaaggg

SEQ ID NO: 10 - DNA, 23 bp, Artificial Sequence
  Description of Artificial Sequence: Synthetic oligonucleotide
ggtgccactaggcacaggagcgg

SEQ ID NO: 11 - DNA, 23 bp, Artificial Sequence
  Description of Artificial Sequence: Synthetic oligonucleotide
tgggccactatggacaggaatgg

SEQ ID NO: 12 - DNA, 23 bp, Artificial Sequence
  Description of Artificial Sequence: Synthetic oligonucleotide
gaggccaccagggacaggctggg

SEQ ID NO: 13 - DNA, 23 bp, Artificial Sequence
  Description of Artificial Sequence: Synthetic oligonucleotide
ggggtacctagggtcaggatggg

SEQ ID NO: 14 - DNA, 20 bp, Artificial Sequence
  Description of Artificial Sequence: Synthetic oligonucleotide
ccggaactctgccctctaac

SEQ ID NO: 15 - DNA, 23 bp, Artificial Sequence
  Description of Artificial Sequence: Synthetic oligonucleotide
agtaggaaagtcccataaggtca

SEQ ID NO: 16 - DNA, 22 bp, Artificial Sequence
  Description of Artificial Sequence: Synthetic oligonucleotide
aagctcatctggtctcccttcc

SEQ ID NO: 17 - DNA, 20 bp, Artificial Sequence
  Description of Artificial Sequence: Synthetic oligonucleotide
tcctgggataccccgaagag

SEQ ID NO: 18 - DNA, 21 bp, Artificial Sequence
  Description of Artificial Sequence: Synthetic oligonucleotide
ctcatggcaagaaagtgctcg

SEQ ID NO: 19 - DNA, 22 bp, Artificial Sequence
  Description of Artificial Sequence: Synthetic oligonucleotide
aattctttgccaaagtgatggg